(19) United States
(12) Patent Application Publication
(10) Pub. No.: US 2018/0036546 A1
(43) Pub. Date: Feb. 8, 2018

(54) WEARABLE DEFIBRILLATORS

(71) Applicant: RAINBOW MEDICAL LTD., Herzliya (IL)

(72) Inventors: Yossi GROSS, Moshav Mazor (IL); Gideon FOSTICK, Givat Shmuel (IL)

(73) Assignee: RAINBOW MEDICAL LTD., Herzliya (IL)

(21) Appl. No.: 15/231,096

(22) Filed: Aug. 8, 2016

(51) Int. Cl. A61N 1/39; A61B 5/00; A61B 5/042; A61B 5/046; A61N 1/04; A61B 5/0408

(52) U.S. Cl. CPC A61N 1/3987; A61N 1/046; A61N 1/0484; A61N 1/3968; A61B 5/04085; A61B 5/0422; A61B 5/046; A61B 5/6805

(57) ABSTRACT

A defibrillator is provided that includes a garment, shaped to be worn around a chest of a patient; cardiac sensing electrodes, each of which is configured for placement in external or internal contact with the chest of the patient; and defibrillation electrodes, each of which is coupled to the garment for external placement near the chest of the patient, and which comprise respective reservoirs containing electrically-conductive gel, and respective liners removably covering the gel. One or more exposure actuators are configured to remove the liners from the gel. Control circuitry is configured to detect cardiac arrhythmia using the cardiac sensing electrodes, ascertain that the detected cardiac arrhythmia is a type of cardiac arrhythmia that can be treated by applying defibrillation, drive the exposure actuators to remove the liners from the gel, and, thereafter, drive the defibrillation electrodes to apply at least one defibrillation shock to the chest of the patient.

### FIELD OF THE APPLICATION

The present invention relates generally to defibrillators, and specifically to wearable defibrillators.

### BACKGROUND OF THE APPLICATION

Heart attacks may cause ventricular fibrillation (VF), which is an arrhythmia characterized by uncoordinated contraction of the cardiac muscle of the ventricles that results in no significant cardiac output. If not treated quickly, typically within minutes, VF generally results in death. Treatment for VF includes prompt cardiopulmonary resuscitation and defibrillation.

Defibrillators deliver a high-voltage shock to the heart to terminate the VF. Some patients with a history of VF are implanted with an automatic implantable cardioverter defibrillator (ICD) that constantly monitors the heart rhythm and applies a shock when necessary. Other patients may be treated using an external defibrillator, such as in a hospital, or an automatic external defibrillator (AED), which is available in some public places.

Still other patients are provided with a wearable external defibrillator, which may comprise a vest, such as if the patients do not qualify for an ICD. For example, post-myocardial infarction patients may need to recover for several months before they can undergo an ICD implantation. Patients may also not be eligible for an ICD if their long-term arrhythmic risk has not yet been established. Such patients can benefit from wearable external defibrillators, which allow them to return to normal life with relative security. The wearable external defibrillator comprises sensing electrodes that sense the patient's electrocardiogram. Upon sensing VF or rapid ventricular tachycardia (VT), the device applies an electrical defibrillation shock to the patient's chest.

### SUMMARY OF THE APPLICATION

Embodiments of the present invention provide a defibrillator that comprises a garment (such as a vest), shaped to be worn around a chest of a patient; cardiac sensing electrodes; defibrillation electrodes; and an external monitor, which comprises control circuitry. Each of the defibrillation electrodes comprises an exposed electrode surface, and is coupled to the garment for external placement of the exposed electrode surface in electrical contact with the chest of the patient for applying one or more defibrillation shocks.

In some applications of the present invention, each of the cardiac sensing electrodes comprises a conductive element and electrical insulation applied to the conductive element so as to electrically insulate the conductive element from direct electrical contact with the skin. The control circuitry is configured to apply a voltage between pairs of the cardiac sensing electrodes, which causes a capacitance-based current flow between the electrodes of each pair.

The control circuitry is configured to detect cardiac arrhythmia using the cardiac sensing electrodes capacitively coupled to the skin, ascertain that the detected cardiac arrhythmia is a type of cardiac arrhythmia that can be treated by applying defibrillation (such as ventricular fibrillation (VF) or rapid ventricular tachycardia (VT)), and, upon ascertaining that the detected cardiac arrhythmia can be treated by applying defibrillation, drive the defibrillation electrodes to apply at least one defibrillation shock to the chest of the patient.

In some applications of the present invention, the defibrillator further comprises one or more external antennas, which are coupled to the garment for placement near the chest of the patient. In addition to or instead of the external cardiac sensing electrodes, the defibrillator comprises intracorporeal cardiac sensing electrode units, which are configured to be placed (e.g., implanted or injected) into the body of the patient, such as under the skin of the patient. Each of the intracorporeal cardiac sensing electrode units comprises (a) a housing; (b) at least two exposed electrode surfaces mounted to the housing; (c) one or more internal antennas mounted to the housing; and (d) electrode-unit circuitry, which is disposed within the housing and configured to wirelessly receive energy using at least one of the one or more internal antennas, sense cardiac signals using the at least two exposed electrode surface, and transmit, using at least one of the one or more internal antennas, signals related to the sensed cardiac signals.

The external control circuitry is configured to (a) transmit energy to the intracorporeal cardiac sensing electrode units using the one or more external antennas, (b) receive, using the one or more external antennas, the signals transmitted by the electrode-unit circuitry of the intracorporeal cardiac sensing electrode units, (c) detect cardiac arrhythmia by analyzing the received signals, (d) ascertain that the detected cardiac arrhythmia is a type of cardiac arrhythmia that can be treated by applying defibrillation (such as VF or rapid VT), and (e) upon ascertaining that the detected cardiac arrhythmia can be treated by applying defibrillation, drive the defibrillation electrodes to apply at least one defibrillation shock to the chest of the patient.

The intracorporeal cardiac sensing electrode units may provide high-quality measurement of the ECG during normal life activity, in which body movement may impede accurate extraction of heart rate parameters. The measurement may be generally continuous, if the external antenna(s) maintains good wireless coupling with each of the intracorporeal cardiac sensing electrode units. Alternatively, the measurement may be intermittent (e.g., occasional and/or unpredictable), such as if the garment and external antenna(s) often move with respect to the chest of the patient, and thus with respect to the intracorporeal cardiac sensing electrode units, during normal life activity. In the latter case, the external control circuitry is generally able to detect life-threatening cardiac arrhythmia even though the intracorporeal cardiac sensing electrode units do not provide continuous cardiac signal detection.

In general, the garment must tightly fit the patient's chest in order for the defibrillation electrodes to achieve good electrical contact with the body. However, a tight fit is inconvenient and uncomfortable for the patient over long periods of time. In some applications of the present invention, the garment is normally worn loose. Immediately before applying one or more defibrillation shocks, the garment is tightened mechanically to achieve close contact between the defibrillation electrodes and the skin. To this end, the defibrillator further comprises a tightening mechanism, which is configured to modulate a tightness of the defibrillation electrodes against the chest of the patient. The control circuitry is configured to (a) detect cardiac arrhythmia using the cardiac sensing electrodes, (b) ascertain that the detected cardiac arrhythmia is a type of cardiac arrhythmia that can be treated by applying defibrillation (such as VF or rapid VT), and (c) upon ascertaining that the detected cardiac arrhythmia can be treated by applying defibrillation, (i) drive the tightening mechanism to increase the tightness of the defibrillation electrodes against the skin of the chest of the patient, and (ii) drive the defibrillation electrodes to apply at least one defibrillation shock to the chest of the patient.

For some applications, the tightening mechanism is configured to modulate the tightness of the defibrillation electrodes against the chest of the patient by modulating a tightness of the garment around the chest of the patient. For some applications, the tightening mechanism comprises one or more balloons, which are configured to push the defibrillation electrodes against the chest of the patient upon inflation of the one or more balloons. For some applications, the tightening mechanism comprises materials that generate a gas, such as CO2, by a chemical reaction. For other applications, the tightening mechanism comprises one or more springs, which are configured to push the defibrillation electrodes against the chest of the patient. Alternatively, the tightening mechanism may comprise one or more springs or electromechanical actuators that are configured to tighten the garment, e.g., by shortening a circumference of a belt-like portion of the garment.

In general, a high-quality measured cardiac signal is necessary to ensure that shocks are applied when necessary (i.e., a low false-negative rate), but only when necessary (i.e., a low false-positive rate). In some applications of the present invention, in order to lower the risk of false positives, the defibrillator further comprises a tightening mechanism, which is configured to modulate a tightness of the cardiac sensing electrodes against the chest of the patient, and the control circuitry is configured to detect the cardiac activity (e.g., an ECG) of the patient using the cardiac sensing electrodes, make a preliminary detection of cardiac arrhythmia based on the detected cardiac activity, and ascertain that the preliminarily-detected cardiac arrhythmia is a type of cardiac arrhythmia that can be treated by applying defibrillation (such as VF or rapid VT).

The control circuitry is configured to, upon ascertaining that the preliminarily-detected cardiac arrhythmia can be treated by applying defibrillation:
drive the tightening mechanism to increase the tightness of the cardiac sensing electrodes against the chest of the patient,
thereafter, again detect the cardiac activity of the patient (e.g., an ECG) using the tightened the cardiac sensing electrodes, and make an improved detection of the cardiac arrhythmia based on the again-detected cardiac activity,
ascertain that the detected cardiac arrhythmia is a type of cardiac arrhythmia that can be treated by applying defibrillation (such as VF or rapid VT), and
upon ascertaining that the cardiac arrhythmia detected by the improved detection can be treated by applying defibrillation, drive the defibrillation electrodes to apply at least one defibrillation shock to the chest of the patient.

For some applications, the tightening mechanism is configured to modulate the tightness of the cardiac sensing electrodes against the chest of the patient by modulating a tightness of the garment around the chest of the patient. For some applications, the defibrillator is configured such that, before the control circuitry drives the tightening mechanism to increase the tightness of the cardiac sensing electrodes against the chest of the patient, the cardiac sensing electrodes only intermittently sense the cardiac activity of the patient, because the cardiac sensing electrodes are not constantly in tight contact with the chest of the patient because of movement of the patient during normal life activity.

For some applications, the tightening mechanism comprises one or more balloons, which are configured to push the cardiac sensing electrodes against the chest of the patient upon inflation of the one or more balloons. For some applications, the tightening mechanism comprises materials that generate a gas, such as CO2, by a chemical reaction.

For some applications, each of defibrillation electrodes further comprises a reservoir containing electrically-conductive gel. In order to release the gel before applying the one or more defibrillation shocks, the defibrillator comprises one or more exposure actuators, which are typically electrically coupled to the control circuitry. Each exposure actuator is configured to remove a liner covering the gel, such as by peeling or sliding the liner off of the gel. For example, each actuator may comprise a motor and a roller. For some applications, one or more disposable single-use cartridges are provided. Each cartridge comprises one or more defibrillation electrodes, liners, reservoir containing gel, and actuators. Each cartridge is inserted into a corresponding receptacle of the garment while the or more liners seal the gel within the one or more reservoirs. After exposure of the gel in conjunction with the application of one or more defibrillation shocks, the cartridge is removed from the garment and replaced with a fresh cartridge.

For some applications, in order to reduce the amplitude or duration of the one or more defibrillation shocks, the control circuitry is configured to apply left-ventricle antitachycardia pacing (ATP) from a passive stimulator implanted in the pericardial space, between the myocardium and the pericardium, or another other cardiac locations, such as in a cardiac chamber, such as in the left or right ventricle. The ATP may be sufficient alone to resolve the fibrillation. If not, the control circuitry applies the one or more defibrillation shocks using the external defibrillation electrodes. The pericardial passive stimulator is powered by and in data communication with the external control circuitry via one or more garment-mounted antennas. For some applications, the ATP device alternatively or additionally functions as a cardiac sensor (e.g., an ECG sensor).

There is therefore provided, in accordance with an application of the present invention, apparatus including a defibrillator, which includes:

(a) a garment, shaped to be worn around a chest of a patient;

(b) cardiac sensing electrodes, each of which is configured for placement in external or internal contact with the chest of the patient to sense cardiac activity of the patient;

(c) defibrillation electrodes, (i) each of which is coupled to the garment for external placement near the chest of the patient for applying one or more defibrillation shocks, and (ii) which include (A) respective reservoirs containing electrically-conductive gel, and (B) respective liners removably covering the gel;

(d) one or more exposure actuators, which are configured to remove the liners from the gel; and

(e) control circuitry, which is configured to:
detect cardiac arrhythmia using the cardiac sensing electrodes,
ascertain that the detected cardiac arrhythmia is a type of cardiac arrhythmia that can be treated by applying defibrillation, and
upon ascertaining that the detected cardiac arrhythmia can be treated by applying defibrillation, (i) drive the one or more exposure actuators to remove the liners from the gel, and, thereafter, (ii) drive the defibrillation electrodes to apply at least one defibrillation shock to the chest of the patient.

For some applications, the one or more exposure actuators are configured to remove the liners from the gel by peeling or sliding the liners off of the gel. For some applications, the one or more exposure actuators include respective motors and rollers.

For some applications, the apparatus further includes one or more disposable single-use cartridges, each of which includes one or more of the defibrillation electrodes, one or more of the liners, one or more of the reservoirs containing the gel, and one or more of the exposure actuators, and the garment is shaped so as to define one or more receptacles for receiving the one or more disposable single-use cartridges.

For some applications, the garment is selected from the group consisting of: a vest and a shirt.

There is further provided, in accordance with an application of the present invention, apparatus including a defibrillator, which includes:

(a) a garment, shaped to be worn around a chest of a patient;

(b) cardiac sensing electrodes, each of which is configured for placement in external or internal contact with the chest of the patient to sense cardiac activity of the patient;

(c) defibrillation electrodes, each of which is coupled to the garment for external placement near the chest of the patient for applying one or more defibrillation shocks;

(d) a tightening mechanism, which is configured to modulate a tightness of the defibrillation electrodes against the chest of the patient; and

(e) control circuitry, which is configured to:
detect cardiac arrhythmia using the cardiac sensing electrodes,
ascertain that the detected cardiac arrhythmia is a type of cardiac arrhythmia that can be treated by applying defibrillation, and
upon ascertaining that the detected cardiac arrhythmia can be treated by applying defibrillation, (i) drive the tightening mechanism to increase the tightness of the defibrillation electrodes against the chest of the patient, and (ii) drive the defibrillation electrodes to apply at least one defibrillation shock to the chest of the patient.

For some applications:

the tightening mechanism is configured to modulate the tightness of the defibrillation electrodes against the chest of the patient by modulating a tightness of the garment around the chest of the patient, and

the control circuitry is configured to, upon ascertaining that the detected cardiac arrhythmia can be treated by applying defibrillation, drive the tightening mechanism to increase the tightness of the garment around the chest of the patient.

For some applications, the tightening mechanism includes one or more balloons, which are configured to push the defibrillation electrodes against the chest of the patient upon inflation of the balloons.

For some applications, the tightening mechanism includes one or more springs.

For some applications:

the defibrillator further includes one or more reservoirs containing electrically-conductive gel, and

the tightening mechanism is configured, when driven to increase the tightness, to release the gel to a region between the defibrillation electrodes and the chest to improve electrical conduction during applying of the at least one defibrillation shock.

For some applications:

the defibrillator further includes one or more reservoirs containing electrically-conductive gel, and

the tightening mechanism is configured, when driven to increase the tightness, to apply pressure to the one or more reservoirs, which releases the gel between the defibrillation electrodes and the chest to improve electrical conduction during applying of the at least one defibrillation shock.

For some applications:

the defibrillation electrodes further include (a) respective reservoirs containing electrically-conductive gel, and (b) respective liners removably covering the gel, and

the defibrillator includes one or more exposure actuators, which are configured to remove the liners from the gel.

For some applications, the one or more exposure actuators are configured to remove the liners from the gel by peeling or sliding the liners off of the gel. For some applications, the one or more exposure actuators include respective motors and rollers.

For some applications, the apparatus further includes one or more disposable single-use cartridges, each of which includes one or more of the defibrillation electrodes, one or more of the liners, one or more of the reservoirs containing the gel, and one or more of the exposure actuators, and the garment is shaped so as to define one or more receptacles for receiving the one or more disposable single-use cartridges.

For some applications, the garment is selected from the group consisting of: a vest and a shirt.

There is still further provided, in accordance with an application of the present invention, apparatus including a defibrillator, which includes:

(a) a garment, shaped to be worn around a chest of a patient;

(b) cardiac sensing electrodes, each of which includes an electrically exposed surface that is coupled to an inner surface of the garment for external placement on skin of the chest of the patient to sense cardiac activity of the patient;

(c) defibrillation electrodes, each of which is coupled to the garment for external placement near the chest of the patient for applying one or more defibrillation shocks;

(d) a tightening mechanism, which is configured to modulate a tightness of the cardiac sensing electrodes against the chest of the patient; and

(e) control circuitry, which is configured to:
detect the cardiac activity of the patient using the cardiac sensing electrodes, make a preliminary detection of cardiac arrhythmia based on the detected cardiac activity, and ascertain that the preliminarily-detected cardiac arrhythmia is a type of cardiac arrhythmia that can be treated by applying defibrillation, and
upon ascertaining that the preliminarily-detected cardiac arrhythmia can be treated by applying defibrillation:
   drive the tightening mechanism to increase the tightness of the cardiac sensing electrodes against the chest of the patient,
   thereafter, again detect the cardiac activity of the patient using the tightened cardiac sensing electrodes, and make an improved detection of the cardiac arrhythmia based on the again-detected cardiac activity,
   ascertain that the detected cardiac arrhythmia is a type of cardiac arrhythmia that can be treated by applying defibrillation, and
   upon ascertaining that the cardiac arrhythmia detected by the improved detection can be treated by applying defibrillation, drive the defibrillation electrodes to apply at least one defibrillation shock to the chest of the patient.

For some applications:

the tightening mechanism is configured to modulate the tightness of the cardiac sensing electrodes against the chest of the patient by modulating a tightness of the garment around the chest of the patient, and

the control circuitry is configured to, upon ascertaining that the preliminarily-detected cardiac arrhythmia can be treated by applying defibrillation, drive the tightening mechanism to increase the tightness of the garment around the chest of the patient.

For some applications, the tightening mechanism includes one or more balloons, which are configured to push the cardiac sensing electrodes against the chest of the patient upon inflation of the balloons. For some applications, the tightening mechanism includes one or more springs.

For some applications, the defibrillator is configured such that, before the control circuitry drives the tightening mechanism to increase the tightness of the cardiac sensing electrodes against the chest of the patient, the cardiac sensing electrodes only intermittently sense the cardiac activity of the patient, because the cardiac sensing electrodes are not constantly in tight contact with the chest of the patient because of movement of the patient during normal life activity.

For some applications:

the defibrillator further includes one or more reservoirs containing electrically-conductive gel, and

the tightening mechanism is configured, when driven to increase the tightness, to release the gel to a region between the cardiac sensing electrodes and the chest to improve electrical conduction when again detecting the cardiac activity using the tightened cardiac sensing electrodes.

For some applications:

the defibrillator further includes one or more reservoirs containing electrically-conductive gel, and

the tightening mechanism is configured, when driven to increase the tightness, to apply pressure to the one or more reservoirs, which releases the gel between the cardiac sensing electrodes and the chest to improve electrical conduction when again detecting the cardiac activity using the tightened cardiac sensing electrodes.

For some applications:

the defibrillation electrodes further include (a) respective reservoirs containing electrically-conductive gel, and (b) respective liners removably covering the gel, and

the defibrillator includes one or more exposure actuators, which are configured to remove the liners from the gel.

For some applications, the one or more exposure actuators are configured to remove the liners from the gel by peeling or sliding the liners off of the gel. For some applications, the one or more exposure actuators include respective motors and rollers.

For some applications, the apparatus further includes one or more disposable single-use cartridges, each of which includes one or more of the defibrillation electrodes, one or more of the liners, one or more of the reservoirs containing the gel, and one or more of the exposure actuators, and the garment is shaped so as to define one or more receptacles for receiving the one or more disposable single-use cartridges.

For some applications, the garment is selected from the group consisting of: a vest and a shirt.

There is additionally provided, in accordance with an application of the present invention, apparatus including an externally-wearable defibrillator, which includes:

(a) a garment, shaped to be worn around a chest of a patient;

(b) cardiac sensing electrodes, each of which (i) is coupled to the garment for external placement on skin of the chest of the patient to sense cardiac activity of the patient, and (ii) includes:
a conductive element; and
electrical insulation applied to the conductive element so as to electrically insulate the conductive element from direct electrical contact with the skin;

(c) defibrillation electrodes, each of which (i) includes an exposed electrode surface, and (ii) is coupled to the garment for external placement of the exposed electrode surface in electrical contact with the chest of the patient for applying one or more defibrillation shocks; and

(d) control circuitry, which is configured to:
detect cardiac arrhythmia using the cardiac sensing electrodes capacitively coupled to the skin,
ascertain that the detected cardiac arrhythmia is a type of cardiac arrhythmia that can be treated by applying defibrillation, and
drive the defibrillation electrodes to apply at least one defibrillation shock to the chest of the patient.

For some applications,

the defibrillation electrodes further include (a) respective reservoirs containing electrically-conductive gel, and (b) respective liners removably covering the gel, and

the defibrillator includes one or more exposure actuators, which are configured to remove the liners from the gel.

For some applications, the one or more exposure actuators are configured to remove the liners from the gel by peeling or sliding the liners off of the gel. For some applications, the one or more exposure actuators include respective motors and rollers.

For some applications, the apparatus further includes one or more disposable single-use cartridges, each of which includes one or more of the defibrillation electrodes, one or more of the liners, one or more of the reservoirs containing the gel, and one or more of the exposure actuators, and the garment is shaped so as to define one or more receptacles for receiving the one or more disposable single-use cartridges.

For some applications, the garment is selected from the group consisting of: a vest and a shirt.

There is yet additionally provided, in accordance with an application of the present invention, apparatus including a defibrillator, which includes:

(a) a garment, shaped to be worn around a chest of a patient;

(b) intracorporeal cardiac sensing electrode units, which are configured to be placed in a body of the patient, and each of which includes:
a housing;
at least two exposed electrode surfaces mounted to the housing;
one or more internal antennas mounted to the housing; and
electrode-unit circuitry, which is disposed within the housing and configured to wirelessly receive energy using at least one of the one or more internal antennas, sense cardiac signals using the at least two exposed electrode surfaces, and transmit, using at least one of the one or more internal antennas, signals related to the sensed cardiac signals;

(c) one or more external antennas, which are coupled to the garment for placement near the chest of the patient;

(d) defibrillation electrodes, each of which is coupled to the garment for external placement near the chest of the patient for applying one or more defibrillation shocks; and

(e) external control circuitry, which is configured to:
transmit energy to the intracorporeal cardiac sensing electrode units using the one or more external antennas,
receive, using the one or more external antennas, the signals transmitted by the electrode-unit circuitry of the intracorporeal cardiac sensing electrode units,
detect cardiac arrhythmia by analyzing the received signals,
ascertain that the detected cardiac arrhythmia is a type of cardiac arrhythmia that can be treated by applying defibrillation, and
drive the defibrillation electrodes to apply at least one defibrillation shock to the chest of the patient.

For some applications, the cardiac sensing electrode units are configured to be implanted subcutaneously.

For some applications, the external control circuitry is configured to detect the cardiac arrhythmia even though wireless coupling between the one or more external antennas and the one or more internal antennas is not continuous because of motion of the garment with respect to the chest of the patient during normal life activity.

For some applications:

the defibrillation electrodes further include (a) respective reservoirs containing electrically-conductive gel, and (b) respective liners removably covering the gel, and

the defibrillator includes one or more exposure actuators, which are configured to remove the liners from the gel.

For some applications, the one or more exposure actuators are configured to remove the liners from the gel by peeling or sliding the liners off of the gel.

For some applications, the one or more exposure actuators include respective motors and rollers.

For some applications, the apparatus further includes one or more disposable single-use cartridges, each of which includes one or more of the defibrillation electrodes, one or more of the liners, one or more of the reservoirs containing the gel, and one or more of the exposure actuators, and the garment is shaped so as to define one or more receptacles for receiving the one or more disposable single-use cartridges.

For some applications, the garment is selected from the group consisting of: a vest and a shirt.

There is further provided, in accordance with an application of the present invention, apparatus for treating a heart of a subject, the apparatus including:

a cardiac stimulator, configured:
to be implanted in the heart of the subject,
to wirelessly receive power, and
to apply a pacing current to the heart of the subject in response to receiving the power; and

control circuitry, coupled to a cardioversion-defibrillation electrode, the control circuitry being configured:
to be implanted subcutaneously in the subject,
to pace the heart by wirelessly driving the cardiac stimulator to apply the pacing current, and
to cardiovert-defibrillate the heart by wiredly driving the cardioversion-defibrillation electrode to apply a cardioverting-defibrillating current to the heart.

For some applications, the control circuitry includes a power-storage element that is configured to power the control circuitry for a continuous period of greater than one hour.

For some applications, the control circuitry includes a power-storage element that is configured to store more than 20 mAh.

For some applications, the cardiac stimulator does not include a power-storage element that is configured to power the cardiac stimulator for a continuous period of greater than one hour.

For some applications, the cardiac stimulator includes a short-term power-storage element that is configured to power the cardiac stimulator for a continuous period of up to one hour.

For some applications, the cardiac stimulator does not include a power-storage element that is configured to store more than 20 mAh.

For some applications, the cardiac stimulator includes a short-term power-storage element that is configured to store up to 20 mAh.

For some applications, the cardioversion-defibrillation electrode is configured to be implanted subcutaneously in the subject.

For some applications, the cardioversion-defibrillation electrode is configured to be implanted in a vicinity of a parasternal midline of the subject.

For some applications, the cardioversion-defibrillation electrode is configured to be implanted in a blood vessel of the subject.

For some applications, the cardioversion-defibrillation electrode is configured to be implanted in a superior vena cava of the subject.

For some applications, the apparatus further includes a cardioversion-defibrillation structure, including the cardioversion-defibrillation electrode; and a cable, including an electrode wire, the cardioversion-defibrillation structure is coupled to the control circuitry via the cable, and the cardioversion-defibrillation electrode is wiredly coupled to the control circuitry via the electrode wire.

For some applications, the cardioversion-defibrillation structure includes at least two cardioversion-defibrillation electrodes, each cardioversion-defibrillation electrode being coupled to the control circuitry via a respective electrode wire.

For some applications, the cardioversion-defibrillation structure is coupled to one end of the cable, and the control circuitry is coupled to another end of the cable.

For some applications, the cable includes a first portion and a second portion, and the cardioversion-defibrillation structure is disposed between the first and second portions of the cable.

There is still further provided, in accordance with an application of the present invention, apparatus for treating a heart of a subject, the apparatus including:

a first device, configured:
to be subcutaneously implanted in the subject,
to detect a factor indicative of a state of the heart of the subject,
to transmit a wireless power signal in response to the detected factor, and
to detect fibrillation of the heart of the subject, and in response to the detected fibrillation, to apply a cardioverting-defibrillating current to tissue of the subject; and

a second device, configured:
to be implanted in the heart of the subject,
to receive the power signal, and
to apply a second current to the heart of the subject, in response to receiving the power signal.

For some applications, the first device includes a power-storage element that is configured to power the first device for a continuous period of greater than one hour.

For some applications, the first device includes a power-storage element that is configured to store more than 20 mAh.

For some applications, the second device does not include a power-storage element that is configured to power the second device for a continuous period of greater than one hour.

For some applications, the second device includes a short-term power-storage element that is configured to power the second device for a continuous period of up to one hour.

For some applications, the second device does not include a power-storage element that is configured to store more than 20 mAh.

For some applications, the second device includes a short-term power-storage element that is configured to store up to 20 mAh.

For some applications, the apparatus further includes:

a cardioversion-defibrillation structure, including a transmitter and a cardioversion-defibrillation electrode; and

at least one cable, the cable including a power-transmitting wire and an electrode wire,

wherein the first device includes control circuitry, the control circuitry:
being wiredly coupled to the cardioversion-defibrillation electrode via the electrode wire,
being configured to drive, via the electrode wire, the cardioversion-defibrillation electrode to apply the cardioverting-defibrillating current,
being wiredly coupled to the transmitter via the power-transmitting wire, and
being configured to drive, via the power-transmitting wire, the transmitter to transmit the wireless power signal.

For some applications:

the first device includes control circuitry and at least one cable, connected to the control circuitry,

the cable includes a power-transmitting wire, and

the first device is configured to transmit the power signal by the control circuitry being configured to drive the power-transmitting wire to transmit the power signal.

For some applications, at least the cable is configured to be subcutaneously implanted transcatheterally.

For some applications, at least the power-transmitting wire is configured to form a loop outside of the control circuitry, the loop being configured to be implanted subcutaneously.

For some applications:

the first device is configured to detect a cardiac cycle of the heart of the subject, and to transmit the power signal in response to the detected cardiac cycle,

the second current includes a pacing current, and

the second device is configured to apply the second current by being configured to apply the pacing current.

For some applications, the first device is configured to detect the cardiac cycle by detecting electrical activity indicative of the cardiac cycle.

For some applications,

the second device includes a magnetic element, configured, when moved in a vicinity of the first device, to induce a third current in the cable, and

the first device is configured to detect the cardiac cycle of the heart by being configured to detect the induced third current.

For some applications, the cable further includes an induction wire, and the magnetic element is configured to induce the third current in the induction wire.

For some applications, the magnetic element is configured to induce the third current in the power-transmitting wire.

For some applications, the power signal includes a first wireless power signal, and the first device is configured to apply the cardioverting-defibrillating current by transmitting a second wireless power signal, the second device being configured to receive the second power signal, and to apply the cardioverting-defibrillating current in response to the second power signal.

For some applications, the apparatus further includes a cardioversion-defibrillation structure, including at least one cardioversion-defibrillation electrode, the cardioversion-defibrillation structure being coupled to the cable and configured to be placed subcutaneously.

For some applications, the cable is configured to form a loop outside of the control circuitry, by the cable and the cardioversion-defibrillation structure being configured to form a loop outside of the control circuitry.

For some applications, the cardioversion-defibrillation structure is configured to be placed in a vicinity of a parasternal midline of the subject.

For some applications, the control circuitry is configured to apply the cardioverting-defibrillating current by being configured to wiredly drive the cardioversion-defibrillation electrode to apply the cardioverting-defibrillating current.

For some applications, the cable includes at least one electrode wire, a first end of the electrode wire being configured to be coupled to the control circuitry, and a second end of the element wire being coupled to the cardioversion-defibrillation electrode.

For some applications:

the cable includes a first portion and a second portion,

the cardioversion-defibrillation structure is disposed between the first and second portions of the cable,

the power-transmitting wire is disposed in both portions of the cable, and

each electrode wire is disposed in one of the portions of the cable, and not in the other portion of the cable.

For some applications:

the at least one electrode includes two electrodes, and

the at least one electrode wire includes two electrode wires, the second end of each electrode wire being coupled to a respective cardioversion-defibrillation electrode, and each electrode wire being disposed in a respective portion of the cable.

For some applications:

the at least one electrode includes two electrodes, and

the at least one electrode wire includes two electrode wires, the second end of each electrode wire being coupled to a respective cardioversion-defibrillation electrode, and both electrode wires being disposed in the same portion of the cable.

For some applications, the second device includes a cardiac stimulator, the cardiac stimulator including:

an electrically-conductive anchor, configured to couple the cardiac stimulator to tissue of the heart of the subject;

an antenna, configured to receive the power signal; and

a controller, configured to apply the second current by driving the anchor to apply the second current to the tissue of the heart of the subject.

For some applications, the cardiac stimulator is configured to be implanted in a chamber of the heart of the subject.

For some applications, the antenna is configured to apply a force to the tissue of the heart of the subject.

For some applications, the antenna has a delivery configuration and a deployed configuration, and is expandable from the delivery configuration to the deployed configuration.

For some applications, the antenna, in the deployed configuration thereof, protrudes from the controller.

For some applications, the cardiac stimulator is configured to detect the factor indicative of the state of the heart of the subject, and to wirelessly transmit a signal indicative of the factor, and the first device is configured to detect the factor indicative of the state of the heart of the subject, by being configured to detect the signal indicative of the factor.

For some applications, the factor indicative of the state of the heart of the subject includes a movement of the heart, and the cardiac stimulator is configured to detect the movement of the heart by being configured to detect movement of the cardiac stimulator when the cardiac stimulator is coupled to the tissue of the heart.

For some applications, the cardiac stimulator is configured to detect the movement of the cardiac stimulator by being configured to detect a change in a received amplitude of the power signal.

For some applications, the factor indicative of the state of the heart of the subject includes an electrical factor of the heart of the subject, and the cardiac stimulator is configured to detect the movement of the heart by being configured to detect the electrical factor.

For some applications, the cardiac stimulator includes a plurality of cardiac stimulators, the plurality of cardiac stimulators including at least a first cardiac stimulator and a second cardiac stimulator.

For some applications:

the first device is configured to transmit a plurality of wireless power signals, the plurality of wireless power signals including at least a first wireless power signal and a second power signal,

the first cardiac stimulator is configured to apply the second current in response to receiving the first wireless power signal, and

the second cardiac stimulator is configured to apply the second current in response to receiving the second wireless power signal.

For some applications, the second cardiac stimulator is configured not to apply the second current in response to receiving the first power signal.

For some applications:

the first device is configured to configure the first and second power signals to have respective first and second characteristics that differ from one another,

the first cardiac stimulator is configured to receive power from the first power signal, in response to an effect of the first characteristic on the first cardiac stimulator, and

the second cardiac stimulator is configured to receive power from the second power signal, in response to an effect of the second characteristic on the second cardiac stimulator.

For some applications:

the first and second characteristics include respective first and second frequencies, and

the first device is configured to configure the first and second power signals to have the respective first and second frequencies.

For some applications:

the first and second characteristics include respective first and second codes, and

the first device is configured to configure the first and second power signals to have the respective first and second codes.

For some applications, the apparatus further includes a third device, configured to be implanted in the body of the subject, and to be in wireless communication with the first device.

For some applications, the third device is configured to wirelessly receive power from the first device.

For some applications:

the third device includes a pressure sensor, configured to be implanted in a pulmonary artery of the subject, to detect a pressure in the pulmonary artery, and to wirelessly transmit a signal indicative of the pressure, and

the first device is configured to transmit the wireless power signal in response to the signal indicative of the detected pressure.

For some applications, the third device includes a tongue-muscle stimulator, configured to be implanted in a vicinity of a tongue muscle of the subject, and the first device is configured to wirelessly drive the tongue-muscle stimulator to apply a current to the tongue muscle of the subject.

There is additionally provided, in accordance with an application of the present invention, apparatus for treating a heart of a subject, the apparatus including:

a leadless pacemaker, configured to be implanted in the heart, and to apply a pacing current to the heart in response to receiving a wireless power signal; and

a subcutaneously-implantable cardioverter-defibrillator (S-ICD), including:
control circuitry; and
a cardioversion-defibrillation structure, including at least one cardioversion-defibrillation electrode, configured to be placed in a vicinity of the heart of the subject,

the control circuitry being configured to:
wiredly drive the cardioversion-defibrillation electrode to apply a cardioverting-defibrillating current to the heart of the subject, and
wirelessly drive the leadless pacemaker to apply the pacing current by transmitting the wireless power signal.

For some applications, the control circuitry includes a power-storage element that is configured to power the control circuitry for a continuous period of greater than one hour.

For some applications, the control circuitry includes a power-storage element that is configured to store more than 20 mAh.

For some applications, the leadless pacemaker does not include a power-storage element that is configured to power the leadless pacemaker for a continuous period of greater than one hour.

For some applications, the leadless pacemaker includes a short-term power-storage element that is configured to power the leadless pacemaker for a continuous period of up to one hour.

For some applications, the leadless pacemaker does not include a power-storage element that is configured to store more than 20 mAh.

For some applications, the leadless pacemaker includes a short-term power-storage element that is configured to store up to 20 mAh.

For some applications, the apparatus further includes a cable, configured to connect the control circuitry to the cardioversion-defibrillation structure, and wherein the cable includes:

a power-transmitting wire, having first and second ends, both ends being configured to be coupled to the control circuitry; and

at least one electrode wire, a first end of the electrode wire being configured to be coupled to the control circuitry, and a second end of the electrode wire being coupled to the cardioversion-defibrillation electrode,

and the control circuitry is configured to wiredly drive the cardioversion-defibrillation electrode via the electrode wire, and

the control circuitry is configured to wirelessly drive the leadless pacemaker by driving the power-transmitting wire to transmit the wireless power signal.

For some applications:

the cable includes a first portion and a second portion,

the cardioversion-defibrillation structure is disposed between the first and second portions of the cable,

the power-transmitting wire is disposed in both portions of the cable, and

each electrode wire is disposed in one of the portions of the cable, and not in the other portion of the cable.

For some applications:

the at least one electrode includes two electrodes, and

the at least one electrode wire includes two electrode wires, the second end of each electrode wire being coupled to a respective cardioversion-defibrillation electrode, and each electrode wire being disposed in a respective portion of the cable.

For some applications:

the at least one electrode includes two electrodes, and

the at least one electrode wire includes two electrode wires, the second end of each electrode wire being coupled to a respective cardioversion-defibrillation electrode, and both electrode wires being disposed in the same portion of the cable.

For some applications, the leadless pacemaker includes:

an electrically-conductive anchor, configured to couple the cardiac stimulator to tissue of the heart of the subject;

an antenna, configured to receive the power signal; and

a controller, configured to apply the second current by driving the anchor to apply the second current to the tissue of the heart of the subject.

For some applications, the leadless pacemaker is configured to be implanted in a chamber of the heart of the subject.

For some applications, the antenna is configured to apply a force to the tissue of the heart of the subject.

For some applications, the antenna has a delivery configuration and a deployed configuration, and is expandable from the delivery configuration to the deployed configuration.

For some applications, the antenna, in the deployed configuration thereof, protrudes from the controller.

For some applications, the leadless pacemaker is configured to detect a factor indicative of a state of the heart of the subject, and to apply the pacing current at least in part responsively to the detected factor.

For some applications:

the leadless pacemaker is configured to wirelessly transmit a signal indicative of the factor, and

the first device is configured to receive the signal indicative of the factor, and to transmit the wireless power signal at least in part responsively to receiving the signal indicative of the factor.

For some applications, the factor indicative of the state of the heart of the subject includes a movement of the heart, and the leadless pacemaker is configured to detect the movement of the heart by being configured to detect movement of the leadless pacemaker when the leadless pacemaker is coupled to the tissue of the heart.

For some applications, the leadless pacemaker is configured to detect the movement of the leadless pacemaker by being configured to detect a change in a received amplitude of the power signal.

For some applications, the factor indicative of the state of the heart of the subject includes an electrical factor of the heart of the subject, and the leadless pacemaker is configured to detect the movement of the heart by being configured to detect the electrical factor.

For some applications, the leadless pacemaker includes a plurality of leadless pacemakers, the plurality of leadless pacemakers including at least:

a first leadless pacemaker, configured to apply the pacing current by being configured to apply a first pacing current, and

a second leadless pacemaker configured to apply the pacing current by being configured to apply a second pacing current.

For some applications:

the S-ICD is configured to transmit a plurality of wireless power signals, the plurality of wireless power signals including at least a first wireless power signal and a second power signal,

the first leadless pacemaker is configured to apply the first pacing current in response to receiving the first wireless power signal, and

the second leadless pacemaker is configured to apply the second pacing current in response to receiving the second wireless power signal.

For some applications, the second leadless pacemaker is configured not to apply the second pacing current in response to receiving the first power signal.

For some applications:

the S-ICD is configured to configure the first and second power signals to have respective first and second characteristics that differ from one another,

the first leadless pacemaker is configured to receive power from the first power signal, in response to an effect of the first characteristic on the first leadless pacemaker, and

the second leadless pacemaker is configured to receive power from the second power signal, in response to an effect of the second characteristic on the second leadless pacemaker.

For some applications:

the first and second characteristics include respective first and second frequencies, and

the first device is configured to configure the first and second power signals to have the respective first and second frequencies.

For some applications:

the first and second characteristics include respective first and second codes, and

the first device is configured to configure the first and second power signals to have the respective first and second codes.

For some applications, the apparatus further includes an additional device, configured to be implanted in the body of the subject, and to be in wireless communication with the S-ICD.

For some applications, the additional device is configured to wirelessly receive power from the S-ICD.

For some applications:

the additional device includes a pressure sensor, configured to be implanted in a pulmonary artery of the subject, to detect a pressure in the pulmonary artery, and to wirelessly transmit a signal indicative of the pressure, and

the S-ICD is configured to transmit the wireless power signal in response to the signal indicative of the detected pressure.

For some applications, the additional device includes a tongue-muscle stimulator, configured to be implanted in a vicinity of a tongue muscle of the subject, and the S-ICD is configured to wirelessly drive the tongue-muscle stimulator to apply a current to the tongue muscle of the subject.

There is yet additionally provided, in accordance with an application of the present invention, apparatus for detecting beating of a heart of a subject, the apparatus including:

an induction wire, configured to be implanted in the subject;

a magnetic element, configured:
to be coupled to the heart of the subject, and
when moved in a vicinity of the induction wire, to induce a current in the induction wire; and

control circuitry, configured:
to be coupled to the induction wire, and
to detect the induced current.

For some applications, the control circuitry includes an implantable cardioverter-defibrillator control circuitry.

For some applications, the apparatus further includes a treatment unit, configured to be coupled to the heart of the subject, the treatment unit including the magnetic element.

For some applications, the treatment unit includes a cardiac pacemaker.

For some applications, the induction wire is configured to be implanted subcutaneously.

For some applications, the induction wire is configured to be implanted around a portion of the subject.

There is also provided, in accordance with an application of the present invention, a method for treating a heart of a subject, the method including:

detecting, using a subcutaneously-implanted first device, fibrillation of the heart of the subject;

in response to the detected fibrillation, applying a cardioverting-defibrillating current to the heart of the subject;

transmitting a wireless power signal using the subcutaneously-implanted first device;

receiving the wireless power signal using a second device that is implanted in the heart; and

in response to receiving the wireless power signal, applying a second current to the heart of the subject using the second device.

For some applications, the second device includes an electrically-conductive anchor, and is coupled to the heart via the electrically-conductive anchor, and applying the second current includes driving the electrically-conductive anchor to apply the second current to the heart.

For some applications, the second device includes an expandable antenna, and receiving the wireless power signal includes receiving the wireless power signal using the expandable antenna of the second device.

For some applications, the first device includes a power-storage element that is configured to power the first device for a continuous period of greater than one hour, and transmitting the wireless power signal includes transmitting the wireless power signal using the first device that includes the power-storage element.

For some applications, the first device includes a power-storage element that is configured to store more than 20 mAh, and transmitting the wireless power signal includes transmitting the wireless power signal using the first device that includes the power-storage element.

For some applications, applying the second current includes applying the second current using a second device that does not include a power-storage element that is configured to power the second device for a continuous period of greater than one hour.

For some applications, the second device includes a short-term power-storage element that is configured to power the second device for a continuous period of up to one hour, and applying the second current includes applying the second current using the second device that includes the short-term power-storage element.

For some applications, applying the second current includes applying the second current using a second device that does not include a power-storage element that is configured to store more than 20 mAh.

For some applications, the second device includes a short-term power-storage element that is configured to store up to 20 mAh, and applying the second current includes applying the second current using the second device that includes the short-term power-storage element.

For some applications, the second device includes a plurality of second devices, and receiving the wireless power signal includes receiving the wireless power signal using the plurality of second devices.

For some applications:

transmitting the wireless power signal includes transmitting a plurality of wireless power signals that includes at least a first wireless power signal and a second wireless power signal, and

receiving the wireless power signal includes receiving at least the first wireless power signal using one of the second devices, and receiving at least the second wireless power signal using another of the second devices.

For some applications, the method further includes not applying the second current to the heart, using the other of the second devices, in response to receiving the first wireless power signal.

For some applications, the plurality of second devices includes at least one cardiac stimulator, and receiving the wireless power signal includes receiving the wireless power signal using the at least one cardiac stimulator.

For some applications, the plurality of second devices includes a plurality of cardiac stimulators, and receiving the wireless power signal includes receiving the wireless power signal using the plurality of cardiac stimulators.

For some applications, the plurality of second devices includes at least one third device that is not a cardiac stimulator, and receiving the wireless power signal includes receiving the wireless power signal using the third device.

For some applications, the third device includes a tongue-muscle stimulator, receiving the wireless power signal using the third device includes receiving the wireless power signal using the tongue-muscle stimulator, and the method further includes, using the tongue-muscle stimulator, applying a current to the tongue of the subject in response to receiving the wireless power signal.

For some applications, the third device includes a blood pressure sensor, receiving the wireless power signal using the third device includes receiving the wireless power signal using the blood pressure sensor, and the method further includes, using the blood pressure sensor, detecting blood pressure of the subject in response to receiving the wireless power signal.

For some applications, the first device includes control circuitry and a cable that includes a power-transmitting wire, coupled to the control circuitry, and transmitting the wireless power signal includes, using the control circuitry, driving the power-transmitting wire to transmit the power signal.

For some applications, the method further includes detecting a cardiac cycle of the heart of the subject, and applying the second current includes applying a pacing current into the heart of the subject in response to detecting the cardiac cycle.

For some applications, detecting the cardiac cycle includes detecting the cardiac cycle using the first device.

For some applications, detecting the cardiac cycle includes detecting the cardiac cycle using the second device.

For some applications, detecting the cardiac cycle includes detecting electrical activity indicative of the cardiac cycle.

For some applications, the second device includes a magnetic element, and detecting the cardiac cycle includes detecting movement of the magnetic element.

For some applications:

the second device includes a magnetic element, configured to induce a third current in the cable, and

detecting the cardiac cycle includes detecting the induced third current.

For some applications, the cable further includes an induction wire, the magnetic element is configured to induce the third current in the induction wire, and detecting the third current includes detecting the third current that is induced in the induction wire.

For some applications, the magnetic element is configured to induce the third current in the power-transmitting wire, and detecting the third current includes detecting the third current that is induced in the power-transmitting wire.

For some applications, applying the cardioverting-defibrillating current includes, using the control circuitry, wiredly driving a cardioversion-defibrillation electrode, connected to the cable, to apply the cardioverting-defibrillating current.

For some applications, applying the cardioverting-defibrillating current includes, using the control circuitry, wirelessly driving the second device to apply the cardioverting-defibrillating current.

There is further provided, in accordance with an application of the present invention, a method for treating a heart of a subject, the method including:

applying a pacing current to the heart of the subject by wirelessly transmitting power from a subcutaneous first device to an intracardiac second device that applies the pacing current in response to the wirelessly-transmitted power; and

applying a cardioverting-defibrillating current into the heart of the subject by wiredly-transmitting power from the subcutaneous first device to a cardioversion-defibrillation structure including at least one cardioversion-defibrillation electrode.

For some applications, applying the pacing current includes applying a plurality of pacing currents to the heart of the subject by wirelessly transmitting power from the subcutaneous first device to a plurality of intracardiac second devices that apply the plurality of pacing currents in response to the wirelessly-transmitted power.

For some applications, the method further includes applying a muscle-contracting current to a tongue muscle of the subject by wirelessly transmitting power from the subcutaneous first device to a sublingual tongue-muscle stimulator that applies the muscle-contracting current in response to the wirelessly-transmitted power.

For some applications, the method further includes detecting blood pressure of the subject by wirelessly transmitting power from the subcutaneous first device to an implanted blood pressure sensor.

For some applications, the method further includes detecting a cardiac cycle of the heart of the subject, and wirelessly transmitting power includes wirelessly transmitting power in response to the detected cardiac cycle.

For some applications, detecting the cardiac cycle includes detecting an electrical signal indicative of the cardiac cycle.

For some applications, detecting the cardiac cycle includes detecting movement of a magnetic element that is coupled to the heart of the subject.

There is still further provided, in accordance with an application of the present invention, a method including:

implanting, in a subject, an induction wire coupled to control circuitry; and

coupling, to a heart of a subject, a magnetic element, such that beating of the heart moves the magnetic element, the magnetic element being configured, when moved, to induce a current in the induction wire,

the control circuitry being configured to detect the beating of the heart by being configured to detect movement of the magnetic element caused by the beating of the heart.

For some applications, coupling the magnetic element to the heart of the subject includes coupling a cardiac stimulator that includes the magnetic element to the heart of the subject.

For some applications, coupling the magnetic element to the heart of the subject includes coupling a plurality of magnetic elements to the heart of the subject.

There is additionally provided, in accordance with an application of the present invention, a method including detecting, using an induction wire implanted in a subject, beating of a heart of the subject, by detecting a current, induced in the induction wire by movement of a magnetic element, the magnetic element being coupled to the heart of the subject such that the beating of the heart moves the magnetic element.

The present invention will be more fully understood from the following detailed description of embodiments thereof, taken together with the drawings, in which:

### BRIEF DESCRIPTION OF THE DRAWINGS

FIGS. 1A-B are schematic front- and back-view illustrations of a defibrillator, in accordance with an application of the present invention;

FIG. 2 is a schematic illustration of an externally-wearable defibrillator, in accordance with an application of the present invention;

FIG. 3 is a schematic illustration of another defibrillator, in accordance with an application of the present invention;

FIGS. 4A-B are schematic illustrations of yet another defibrillator, in accordance with an application of the present invention;

FIGS. 5A-B are schematic illustrations of a still another defibrillator, in accordance with an application of the present invention;

FIG. 6 is a schematic illustration of a gel-releasing configuration of defibrillation electrodes of the defibrillator of FIGS. 1A-B, in accordance with an application of the present invention;

FIGS. 7A-C are schematic cross-sectional illustrations of the gel-releasing configuration of the defibrillation electrodes of FIG. 6, in accordance with an application of the present invention;

FIGS. 8A-B are schematic illustrations of disposable single-use cartridges and the defibrillator of FIGS. 1A-B, in accordance with an application of the present invention;

FIGS. 9A-C are schematic illustrations of a cardiac stimulator, and steps in the implantation thereof, in accordance with some applications of the invention;

FIGS. 10A-B are schematic illustrations of heart-monitoring apparatus comprising a magnetic element and a detector, in accordance with some applications of the invention; and

FIG. 11 is a schematic illustration of apparatus for pacing and cardioverting-defibrillating a heart of a subject, in accordance with some applications of the invention.

### DETAILED DESCRIPTION OF APPLICATIONS

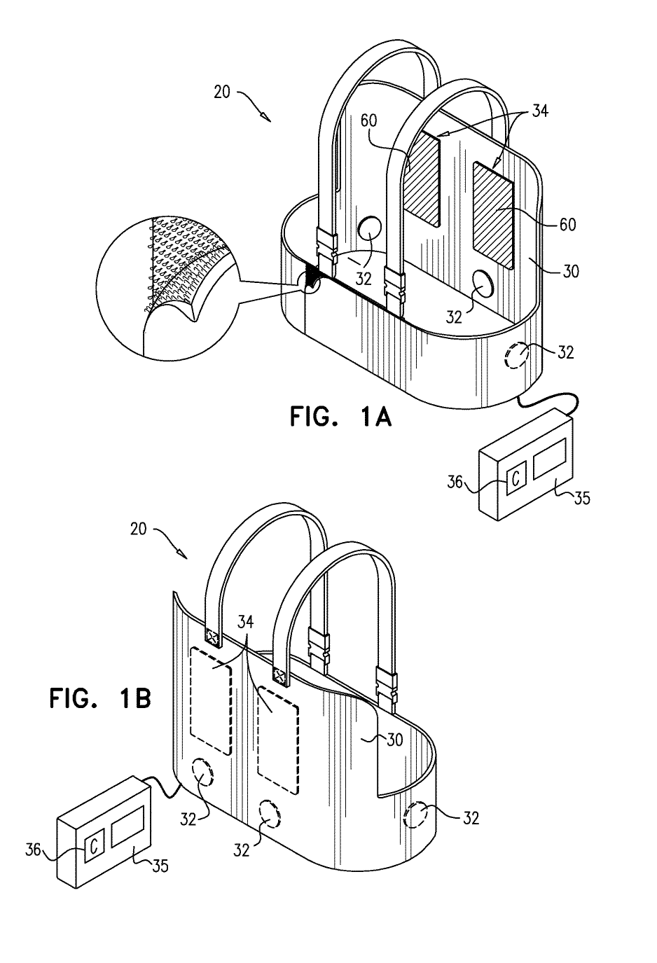

FIGS. 1A-B are schematic front- and back-view illustrations of a defibrillator 20, in accordance with an application of the present invention. Defibrillator 20 comprises a garment 30, shaped to be worn around a chest of a patient; cardiac sensing electrodes 32; defibrillation electrodes 34; and an external monitor 35, which comprises control circuitry 36, which typically comprises appropriate memory, processor(s), and hardware running software that is configured to provide the functionality of the control circuitry described herein. As used in the present application, including in the claims, a “garment” is any article configured to be worn; a garment does not necessarily comprise fabric, but may instead comprise only straps, for example. For some applications, garment 30 comprises a vest, such as shown in the figures. Alternatively, garment 30 may comprise a shirt, such as an undershirt, or another type of garment with any shape or design (configurations not shown).

Each of defibrillation electrodes 34 comprises an exposed electrode surface 60, and is coupled to garment 30 for external placement of exposed electrode surface 60 in electrical contact with the chest of the patient for applying one or more defibrillation shocks. Typically, at least one (e.g., two) of defibrillation electrodes 34 is disposed at the back of garment 30, for placement against the back of the patient. Optionally, at least one (e.g., one or two) of defibrillation electrodes 34 is disposed at the front of garment 30, for placement against the front of the patient's chest, and/or is disposed at one or both sides of garment 30, for placement against one or both sides of the patient's chest.

For some applications, such as in which garment 30 comprises a shirt, cardiac sensing electrodes 32 comprise conductive fibers that are woven into the fabric of the garment.

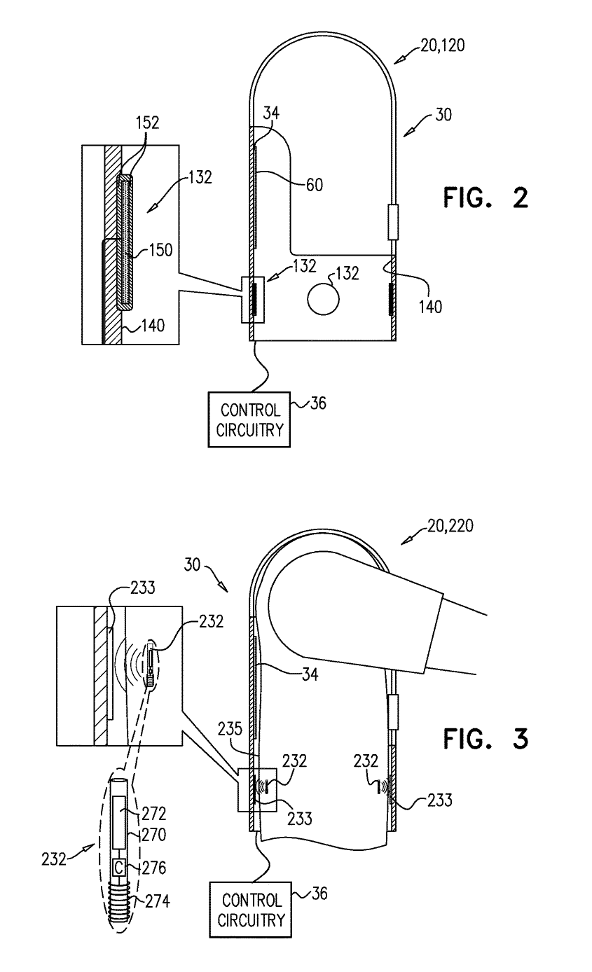

Reference is made to FIG. 2, which is a schematic illustration of an externally-wearable defibrillator 120, in accordance with an application of the present invention. Externally-wearable defibrillator 120 is one implementation of defibrillator 20, described hereinabove with reference to FIGS. 1A-B. Defibrillator 120 comprises garment 30; cardiac sensing electrodes 132; defibrillation electrodes 34; and external monitor 35, which comprises control circuitry 36. Each of cardiac sensing electrodes 132 is coupled to garment 30 for external placement on skin of the chest of the patient to sense cardiac activity of the patient (e.g., an electrocardiogram (ECG)). For some applications, cardiac sensing electrodes 132 are embedded within fabric of garment 30, which increases comfort for the patient. For other applications, cardiac sensing electrodes 132 are coupled to an inner surface 140 of garment 30.

Each of cardiac sensing electrodes 132 comprises a conductive element 150, and electrical insulation 152 (e.g., silicone) applied to conductive element 150 so as to electrically insulate the conductive element from direct electrical contact with the skin. Control circuitry 36 is configured to sense a voltage between pairs of cardiac sensing electrodes 132 that causes a capacitance-based current flow between the electrodes of each pair. In other words, current does not flow directly from the electrodes to the skin; rather, electric fields in the skin capacitively drive a current in the electrodes.

Control circuitry 36 is configured to detect cardiac arrhythmia using cardiac sensing electrodes 132 capacitively coupled to the skin (e.g., using a differential gain amplifier), ascertain that the detected cardiac arrhythmia is a type of cardiac arrhythmia that can be treated by applying defibrillation (such as ventricular fibrillation (VF) or rapid ventricular tachycardia (VT)), and, upon ascertaining that the detected cardiac arrhythmia can be treated by applying defibrillation, drive defibrillation electrodes 34 to apply at least one defibrillation shock to the chest of the patient.

The use of capacitive cardiac sensing electrodes 132 may overcome contact issues and motion artifacts that often occur when using conventional external ECG sensing electrodes. In addition, capacitive cardiac sensing electrodes 132 do not require skin preparation and are generally insensitive to skin conditions. Furthermore, the use of capacitive cardiac sensing electrodes 132 may increase the water-resistance of defibrillator 120 (such as for use in a shower or bath), because the use of capacitive coupling reduces the possibility of a short circuit of the sensing electrodes.

For some applications, cardiac sensing electrodes 132 comprise EPIC sensors (Plessey Semiconductors Ltd., UK).

Reference is now made to FIG. 3, which is a schematic illustration of a defibrillator 220, in accordance with an application of the present invention. Defibrillator 220 is one implementation of defibrillator 20, described hereinabove with reference to FIGS. 1A-B. Defibrillator 220 comprises garment 30; intracorporeal cardiac sensing electrode units 232; defibrillation electrodes 34; and external monitor 35, which comprises control circuitry 36. Defibrillator 220 further comprises one or more external antennas 233, which are coupled to garment 30 for placement near the chest of the patient; for example, each of the one or more antennas may comprise a coil. For some applications, each of the one or more antennas comprise one or more coils that are looped circumferentially around garment 30, such that the one or more coils encircle the patient's chest when wearing the garment, which may increase the efficiency of the antenna(s). The loops of the coil(s) may be permanently closed (for example, for applications in which the garment comprises an undershirt), or may comprise a closure mechanism.

Intracorporeal cardiac sensing electrode units 232 are configured to be placed (e.g., implanted or injected) into the body of the patient, such as under skin 235 of the patient (i.e., subcutaneously). Each of intracorporeal cardiac sensing electrode units 232 comprises:
a housing 270;
at least two exposed electrode surfaces 272 mounted to housing 270;
one or more internal antennas 274 mounted to housing 270; and
electrode-unit circuitry 276, which is disposed within housing 270 and configured to wirelessly receive energy (either intermittently or generally continuously) using at least one of the one or more internal antennas 274, sense cardiac signals (either intermittently or generally continuously) using the at least two exposed electrode surfaces 272, and transmit, using at least one of the one or more internal antennas 274, signals related to the sensed cardiac signals.

For some applications, housing 270 is shaped generally as a capsule, e.g., is generally cylindrical; for example, housing 270 may have a diameter of at least 1 mm, no more than 5 mm, and/or between 1 and 5 mm, and a length of at least 10 mm, no more than 80 mm, and/or between 10 and 80 mm. For some applications, housing 270 is configured to be injected transcutaneously, for example using a hollow needle in a simple, non-surgical procedure. For some applications, each of intracorporeal cardiac sensing electrode units 232 comprises a power storage element, such as a capacitor (although the units typically do not comprise batteries).

External control circuitry 36 is configured to:
transmit energy to intracorporeal cardiac sensing electrode units 232 using the one or more external antennas 233 (transceivers),
receive, using the one or more external antennas 233, the signals transmitted by electrode-unit circuitry 276 of intracorporeal cardiac sensing electrode units 232,
detect cardiac arrhythmia by analyzing the received signals (for example, by generating an ECG based on the received signals),
ascertain that the detected cardiac arrhythmia is a type of cardiac arrhythmia that can be treated by applying defibrillation (such as VF or rapid VT), and
upon ascertaining that the detected cardiac arrhythmia can be treated by applying defibrillation, drive defibrillation electrodes 34 to apply at least one defibrillation shock to the chest of the patient.

For some applications, the power is transmitted wirelessly to intracorporeal cardiac sensing electrode units 232 using induction (typically radiofrequency). For some applications, external control circuitry 36 and electrode-unit circuitry 276 implement near-field communication (NFC)-type power supply and communication (NFC allows compatible hardware to both supply power to and communicate with an otherwise unpowered and passive electronic module using radio waves). Upon entering the externally-generated RF field, cardiac sensing electrode units 232 are able to draw enough power from the field to activate internal amplifiers and transmit acquired data. When cardiac sensing electrode units 232 draw power in this way, the resultant interaction of the RF fields causes the voltage at the transceiver antenna to drop in value. This effect is utilized by cardiac sensing electrode units 232 to communicate information to external control circuitry 36. Cardiac sensing electrode units 232 are able to control the amount of power drawn from the field, and by doing so they can modulate the voltage sensed at the external transceiver according to the data stream being transmitted.

For some applications, intracorporeal cardiac sensing electrode units 232 comprise a directionally orthogonal set of contacts for multi-channel ECG recording, thus leveraging high correspondence (positive or negative correlation) of orthogonal ECG projections due to the sharp nature of the QRS complex, while the superimposed noise is of a typically more random nature. This will allow separation of correlated from uncorrelated data using statistical methods such as principal component analysis (PCA) or independent component analysis (ICA), thus effectively enhancing the signal-to-noise ratio of the intercepted signals and thereby facilitating high quality analysis.

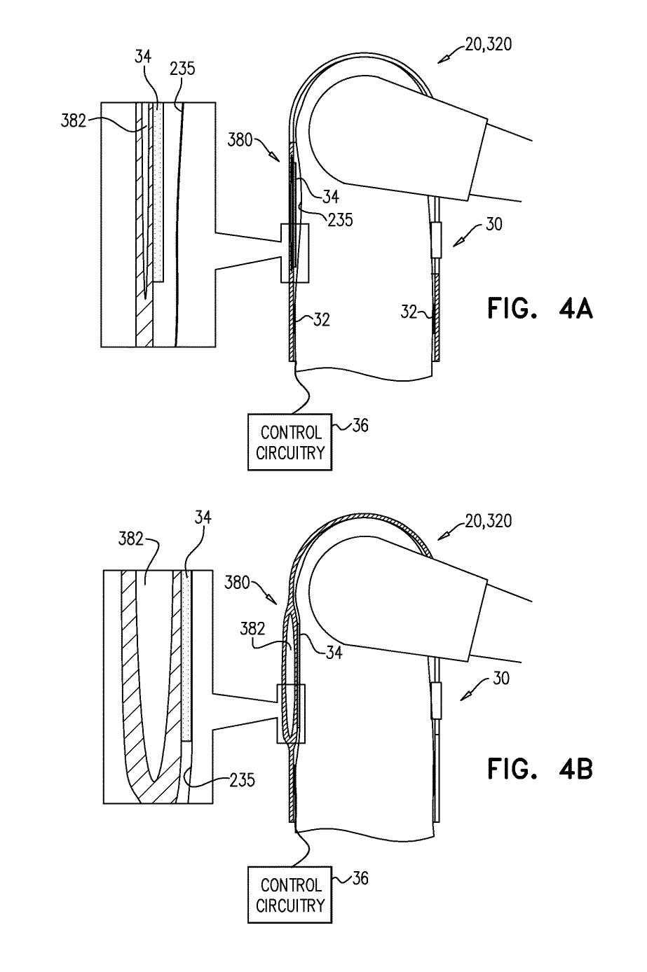

Reference is now made to FIGS. 4A-B, which are schematic illustrations of a defibrillator 320, in accordance with an application of the present invention. Defibrillator 320 is one implementation of defibrillator 20, described hereinabove with reference to FIGS. 1A-B. The features of defibrillator 320 may be combined with those of defibrillator 120 and/or defibrillator 220, described hereinabove with reference to FIGS. 2 and 3, respectively, mutatis mutandis. Defibrillator 320 comprises garment 30; cardiac sensing electrodes 32, each of which is configured for placement in external or internal contact with the chest of the patient to sense cardiac activity of the patient (e.g., an ECG); defibrillation electrodes 34; and control circuitry 36.

In general, garment 30 must tightly fit the patient's chest in order for defibrillation electrodes 34 to achieve good electrical contact with the body. However, a tight fit is inconvenient and uncomfortable for the patient over long periods of time. In some applications of the present invention, garment 30 is normally worn loose, such as shown in FIG. 4A. Before (typically, immediately before) applying one or more defibrillation shocks, garment 30 is tightened mechanically to achieve close contact between defibrillation electrodes 34 and skin 235, such as shown in FIG. 4B. To this end, defibrillator 320 further comprises a tightening mechanism 380, which is configured to modulate a tightness of defibrillation electrodes 34 against the chest of the patient. Control circuitry 36 is configured to:
detect cardiac arrhythmia using cardiac sensing electrodes 32,
ascertain that the detected cardiac arrhythmia is a type of cardiac arrhythmia that can be treated by applying defibrillation (such as VF or rapid VT), and
upon ascertaining that the detected cardiac arrhythmia can be treated by applying defibrillation, (i) drive tightening mechanism 380 to increase the tightness of defibrillation electrodes 34 against skin 235 of the chest of the patient, and (ii) drive defibrillation electrodes 34 to apply at least one defibrillation shock to the chest of the patient.

For some applications, tightening mechanism 380 is configured to modulate the tightness of defibrillation electrodes 34 against the chest of the patient by modulating a tightness of garment 30 around the chest of the patient. Control circuitry 36 is configured to, upon ascertaining that the detected cardiac arrhythmia can be treated by applying defibrillation, drive tightening mechanism 380 to increase the tightness of garment 30 around the chest of the patient.

For some applications, tightening mechanism 380 comprises one or more balloons 382, which are configured to push defibrillation electrodes 34 against the chest of the patient upon inflation of the one or more balloons 382. For some applications, tightening mechanism 380 comprises materials that generate a gas, such as CO2, by a chemical reaction.

For some applications, tightening mechanism 380 comprises one or more springs, which are configured to push defibrillation electrodes 34 against the chest of the patient (configuration not shown). For example, the springs may be held compressed (e.g., by respective latches) until triggered by control circuitry 36 (e.g., via a drawstring). Alternatively, tightening mechanism 380 may comprise one or more springs or electromechanical actuators that are configured to tighten garment 30, e.g., by shortening a circumference of a belt-like portion of garment 30. For example, the springs may be held compressed (e.g., by respective latches) until triggered by control circuitry 36 (e.g., via a drawstring).

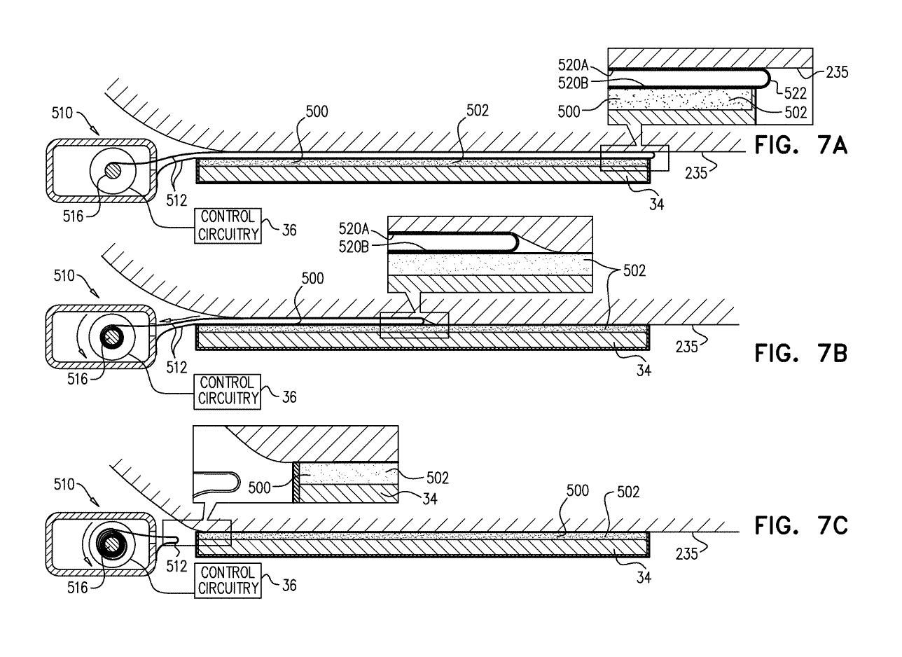

For some applications, defibrillator 320 further comprises one or more reservoirs containing electrically-conductive gel. Tightening mechanism 380 is configured, when driven to increase the tightness, to release the gel to a region between defibrillation electrodes 34 and the chest to improve electrical conduction during applying of the at least one defibrillation shock. Alternatively or additionally, tightening mechanism 380 is configured, when driven to increase the tightness, to apply pressure to the reservoirs, which releases the gel between the defibrillation electrodes and the chest to improve electrical conduction during applying of the at least one defibrillation shock. Further alternatively or additionally, defibrillator 320 implements the gel-releasing configuration described hereinbelow with reference to FIGS. 6 and 7A-C.

For some applications, cardiac sensing electrodes 32 comprise respective exposed electrode surfaces for placement against an external surface of skin 235, as shown in FIGS. 4A-B. For other applications, cardiac sensing electrodes 32 comprise respective intracorporeal cardiac sensing electrode units 232, as described hereinabove with reference to FIG. 3. For still other applications, cardiac sensing electrodes 32 comprise respective capacitive cardiac sensing electrodes 132, described hereinabove with reference to FIG. 2.

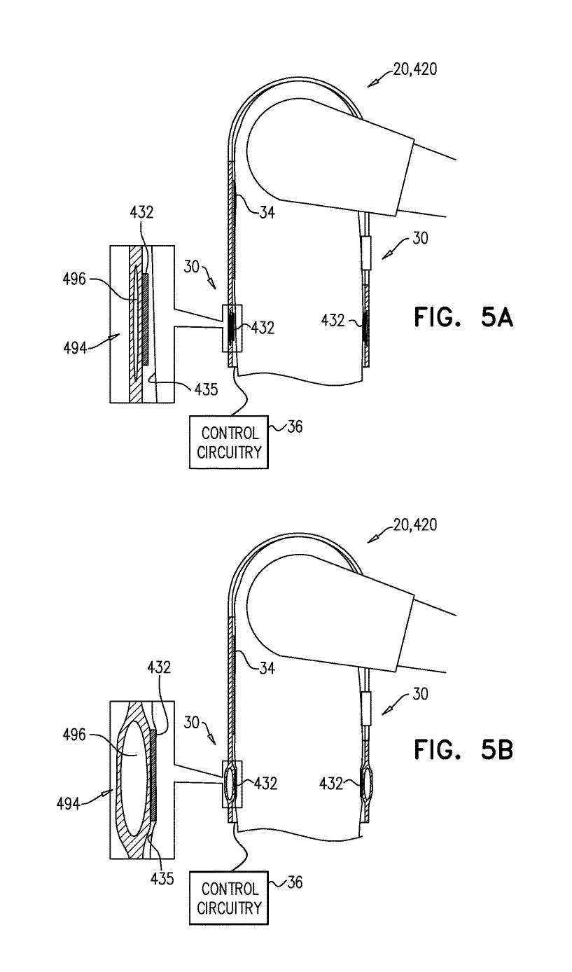

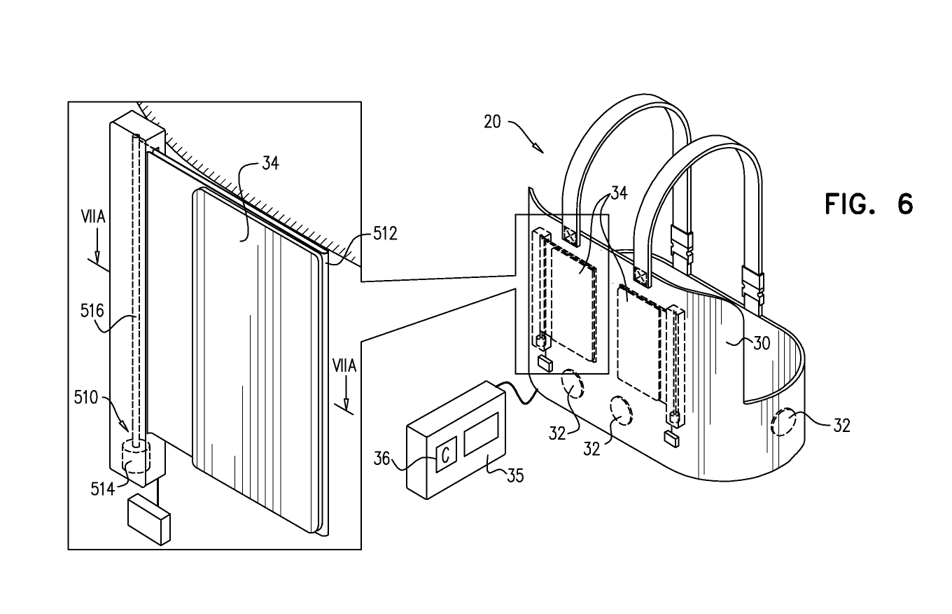

Reference is now made to FIGS. 5A-B, which are schematic illustrations of a defibrillator 420, in accordance with an application of the present invention. Defibrillator 420 is one implementation of defibrillator 20, described hereinabove with reference to FIGS. 1A-B. The features of defibrillator 420 may be combined with those of defibrillator 120, defibrillator 220, and/or defibrillator 320, described hereinabove with reference to FIGS. 2, 3, and 4A-B, respectively, mutatis mutandis. Defibrillator 420 comprises garment 30; cardiac sensing electrodes 432; defibrillation electrodes 34; and control circuitry 36. Typically, each of cardiac sensing electrodes 432 comprises an electrically exposed surface that is coupled to an inner surface 440 of garment 30 for external placement on skin 235 of the chest of the patient to sense cardiac activity of the patient (e.g., an ECG).

In general, a high-quality measured cardiac signal is necessary to ensure that shocks are applied when necessary (i.e., a low false-negative rate), but only when necessary (i.e., a low false-positive rate). In some applications of the present invention, in order to lower the risk of false positives, defibrillator 420 further comprises a tightening mechanism 494, which is configured to modulate a tightness of cardiac sensing electrodes 432 against the chest of the patient, and control circuitry 36 is configured to:
detect the cardiac activity (e.g., an ECG) of the patient using cardiac sensing electrodes 432, make a preliminary detection of cardiac arrhythmia based on the detected cardiac activity, and ascertain that the preliminarily detected-cardiac arrhythmia is a type of cardiac arrhythmia that can be treated by applying defibrillation (such as VF or rapid VT), and
upon ascertaining that the preliminarily-detected cardiac arrhythmia can be treated by applying defibrillation:
   drive tightening mechanism 494 to increase the tightness of cardiac sensing electrodes 432 against the chest of the patient,
   thereafter, again detect the cardiac activity of the patient (e.g., an ECG) using the tightened cardiac sensing electrodes 432, and make an improved detection of the cardiac arrhythmia based on the again-detected cardiac activity,
   ascertain that the detected cardiac arrhythmia is a type of cardiac arrhythmia that can be treated by applying defibrillation (such as VF or rapid VT), and
   upon ascertaining that the cardiac arrhythmia detected by the improved detection can be treated by applying defibrillation, drive defibrillation electrodes 34 to apply at least one defibrillation shock to the chest of the patient.

For some applications, tightening mechanism 494 is configured to modulate the tightness of cardiac sensing electrodes 432 against the chest of the patient by modulating a tightness of garment 30 around the chest of the patient. Control circuitry 36 is configured to, upon ascertaining that the preliminarily detected-cardiac arrhythmia can be treated by applying defibrillation, drive tightening mechanism 494 to increase the tightness of garment 30 around the chest of the patient. For some applications, control circuitry 36 is configured to reduce the tightness of garment 30 around the chest of the patient upon ascertaining that the preliminarily-detected cardiac arrhythmia was not in fact cardiac arrhythmia (and/or, optionally, that the cardiac arrhythmia cannot be treated by applying defibrillation).

For some applications, tightening mechanism 494 comprises one or more balloons 496, which are configured to push cardiac sensing electrodes 432 against the chest of the patient upon inflation of the one or more balloons 496. For some applications, tightening mechanism 494 comprises materials that generate a gas, such as CO2, by a chemical reaction.

For some applications, tightening mechanism 494 comprises one or more springs, which are configured to push cardiac sensing electrodes 432 against the chest of the patient (configuration not shown). For example, the springs may be held compressed (e.g., by respective latches) until triggered by control circuitry 36 (e.g., via a drawstring). Alternatively, tightening mechanism 494 may comprise one or more springs or electromechanical actuators that are configured to tighten garment 30, e.g., by shortening a circumference of a belt-like portion of garment 30. For example, the springs may be held compressed (e.g., by respective latches) until triggered by control circuitry 36 (e.g., via a drawstring).

For some applications, defibrillator 420 further comprises one or more reservoirs containing electrically-conductive gel. For some of these applications, tightening mechanism 494 is configured, when driven to increase the tightness, to release the gel to regions between cardiac sensing electrodes 432 and the chest to improve electrical conduction when again detecting the cardiac activity after tightening cardiac sensing electrodes 432. Alternatively or additionally, tightening mechanism 494 is configured, when driven to increase the tightness, to apply pressure to the reservoirs, which releases the gel between the defibrillation electrodes and the chest to improve electrical conduction during applying of the at least one defibrillation shock.

Reference is made to FIGS. 6 and 7A-C, which are schematic illustrations of gel-releasing configuration of defibrillation electrodes 34, in accordance with an application of the present invention. FIGS. 7A-C are cross-sectional views of FIG. 6 (with FIG. 7A taken along line VIIA-VIIA of FIG. 6). This gel-releasing configuration can be used in combination with any of the configurations of defibrillator 20 described hereinabove with reference to FIGS. 1A-5. For some applications, each of defibrillation electrodes 34 further comprises a reservoir 500 containing electrically-conductive gel 502 (shown in FIGS. 7A-B). In order to release gel 502 before applying the one or more defibrillation shocks, defibrillator 20 comprises one or more exposure actuators 510 (such as one exposure actuator 510 per defibrillation electrode 34), which are typically electrically coupled to control circuitry 36. Control circuitry 36 is configured to activate the one or more exposure actuators 510 to release gel 502 before driving defibrillation electrodes 34 to apply the at least one defibrillation shock to the chest of the patient. Each exposure actuator 510 is configured to remove a liner 512 removably covering gel 502, such as by peeling or sliding liner 512 off of the gel. For example, each actuator may comprise a motor 514 and a roller 516, which may use rotation for removing the liner.

For some applications, liner 512 is initially folded so as to define two portions, first and second portions 520A and 520B, alongside each other, which meet at a distal fold 522. Second portion 520B directly contacts gel 502, and first portion 520A does not directly contact gel 502, but instead is exposed for placement against skin 235. Typically, second portion 520B is removably sealed around the perimeter of reservoir 500 (e.g., by RF, glue, or heat), in order to prevent leakage of gel 502. Exposure actuator 510 is arranged to pull first portion 520A proximally (to the left in FIGS. 7A-C). First portion 520A in turn pulls second portion 520B proximally, peeling second portion 520B off of gel 502. This folded arrangement results in second portion 520B effectively being pulled from its distal (far) end, rather than its proximal (close) end, which may aid in overcoming friction between the skin and the liner during removal of the liner.

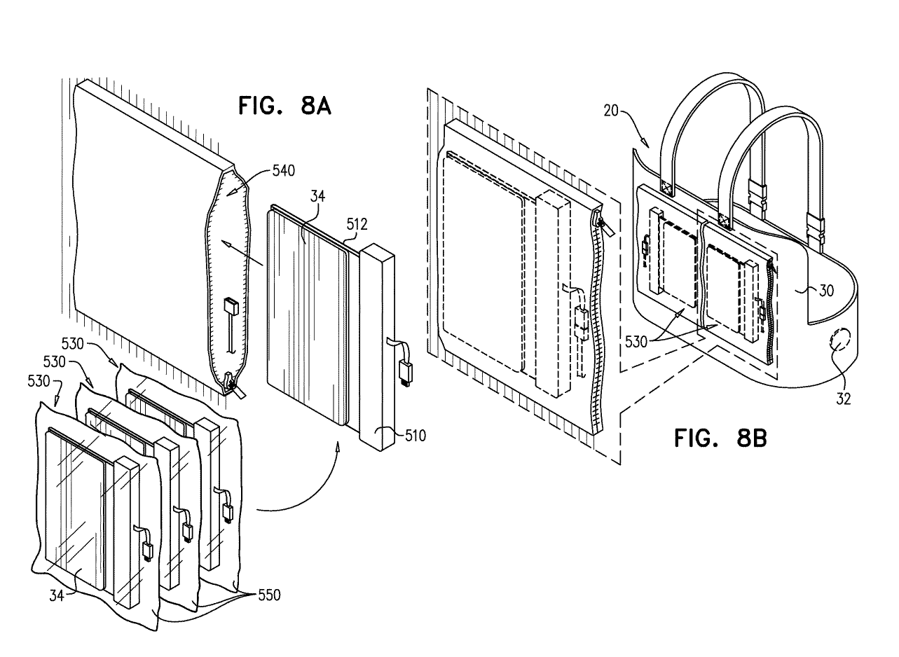

Reference is now made to FIGS. 8A-B, which are schematic illustrations of disposable single-use cartridges 530 and defibrillator 20, in accordance with an application of the present invention. Each cartridge 530 comprises one or more defibrillation electrodes 34, liners 512, reservoir 500 containing gel 502, and exposure actuators 510. Each cartridge 530 is inserted into a corresponding receptacle 540 of garment 30 while the or more liners 512 seal gel 502 within the one or more reservoirs 500. After exposure of gel 502 in conjunction with the application of one or more defibrillation shocks, cartridge 530 is removed from the garment and replaced with a fresh cartridge 530. Typically, each cartridge 530 is provided in separate packaging 550, typically sterile packaging.

Alternatively or additionally, the defibrillator may comprise a tightening mechanism, such as described hereinabove, for releasing the gel.

Reference is made to FIGS. 1A-5B. For some applications, in order to reduce the amplitude or duration of the one or more defibrillation shocks, the control circuitry is configured to apply left-ventricle antitachycardia pacing (ATP) from a passive stimulator implanted in the pericardial space, between the myocardium and the pericardium, or another other cardiac locations, such as in a cardiac chamber, such as in the left or right ventricle. The ATP may be sufficient alone to resolve the fibrillation. If not, the control circuitry applies the one or more defibrillation shocks using the external defibrillation electrodes. The pericardial passive stimulator is powered by and in data communication with the external control circuitry via one or more garment-mounted antennas (for example, external antennas 233, described hereinabove with reference to FIG. 3, and/or by one or more dedicated antennas). Alternatively, the pericardial implant comprises a battery. Alternatively, the ATP device is configured to be placed elsewhere, typically in physical contact with the heart. For some applications, the ATP device alternatively or additionally functions as a cardiac sensor (e.g., an ECG sensor).

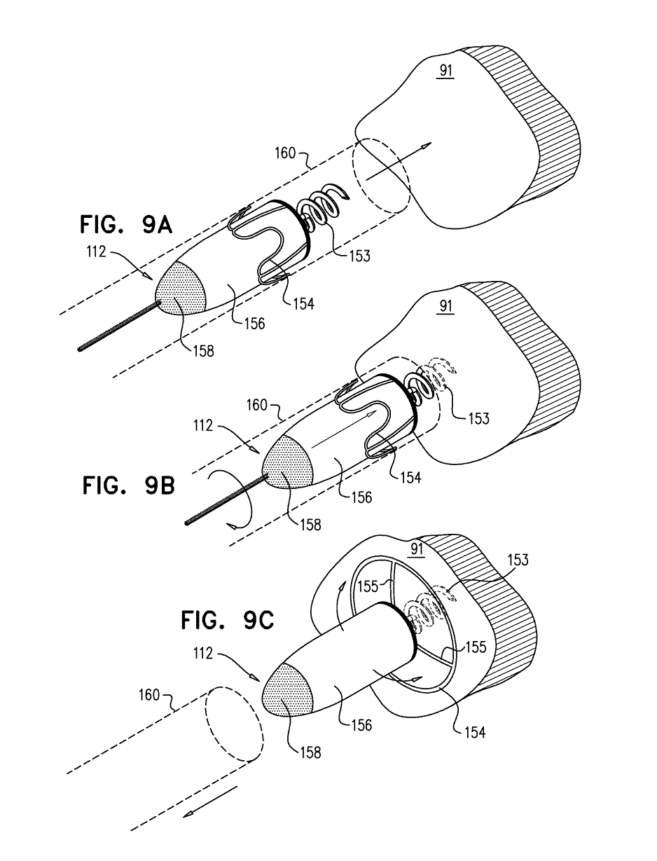

Reference is made to FIGS. 9A-C, which are schematic illustrations of cardiac stimulator 112, and steps in the implantation thereof at an implantation site, in accordance with some applications of the invention. Stimulator 112 comprises an electrically-conducting anchor 153, an antenna 154, and a controller 156. Anchor 153 is configured to couple stimulator 112 to tissue 91 of the heart of the subject, and is typically disposed at one end of controller 156. Anchor 153 typically has a corkscrew shape, and is configured to be screwed into tissue 91, and to act as an electrode (e.g., as a first electrode). Stimulator 112 further comprises a second electrode 158, typically disposed at the other end of controller 156 to anchor 153. Typically, stimulator 112 is configured such that anchor 153 acts as a cathode and second electrode 158 acts as an anode (e.g., controller 156 is configured such that anchor 153 acts as a cathode and second electrode 158 acts as an anode). Antenna 154 typically comprises a plurality of struts 155, and is typically expandable from a constrained delivery configuration toward a deployed configuration, as described hereinbelow.

Stimulator 112 is typically configured to wirelessly receive power and/or other wireless signals using antenna 154 (e.g., power from medical device 96, as described hereinabove, and/or power from a subcutaneous device 110 or 210, as described hereinbelow). As described hereinabove, stimulator 112 typically comprises no power-storage element (i.e., no power-storage element that is configured to power the stimulator for a continuous period of greater than 1 hour and/or to store more than 20 mAh). Stimulator 112 thereby typically consumes power as it receives the power. For some applications of the invention, stimulator 112 does comprise a power-storage element. For some applications of the invention, stimulator 112 comprises a short-term power-storage element, and is configured to continue applying current to the heart of the subject during periods that the stimulator does not receive the power. For example, stimulator 112 may be configured to continue to function during replacement of the device from which the stimulator receives power (e.g., during replacement of device 96, 110 or 210 and/or power-storage elements thereof). Typically, the short-term power-storage element is configured to power the stimulator for a continuous period of up to 1 hour and/or to store up to 20 mAh.

For some applications of the invention, antenna 154 comprises a rectifying antenna, and is configured to receive power via RF signals. For some applications of the invention, antenna 154 comprises an induction coil, and is configured to receive power via magnetic induction.

Typically, controller 156 is encapsulated (e.g., hermetically sealed) in a casing (not shown). The casing may comprise any material suitable for implantation in the subject and for protection of the controller, but typically comprises ceramic, glass, and/or metal (e.g., titanium). Anchor 153, antenna 154, and second electrode 158 typically protrude through the casing, and/or are wiredly coupled to the controller via respective wires that protrude through the casing.

FIG. 9A shows cardiac stimulator 112 having been delivered to the heart of the subject, and placed in contact with tissue 91 of the heart (e.g., within a ventricle or atrium of the heart). Typically, stimulator 112 is configured to be delivered percutaneously (e.g., transluminally). As shown in FIG. 9A, stimulator 112 is typically delivered while antenna 154 is in the constrained delivery configuration thereof, within an overtube 160, which constrains the stimulator in the constrained configuration. For some applications, overtube 160 comprises a 6 Fr catheter. For some applications, overtube 160 (e.g., a portion thereof) is itself advanced to the heart transcatheterally. In the constrained configuration, antenna 154 is typically but not necessarily disposed against controller 156 (e.g., against lateral sides thereof).

To couple stimulator 112 to tissue 91, anchor 153 is typically rotated (typically by rotating all of stimulator 112), as shown in FIG. 9B. Once stimulator 112 is coupled to tissue 91, overtube 160 is removed from the stimulator (e.g., by sliding the overtube proximally), as shown in FIG. 9C. Typically, antenna 154 is configured to automatically move toward the deployed configuration thereof when overtube 160 is removed, as shown in FIG. 9C. In the deployed configuration, antenna 154 (e.g., portions thereof) typically protrudes radially from controller 156. It is hypothesized that the expansion of antenna 154 from the delivery configuration to the deployed configuration facilitates reception and/or transmission of wireless signals (e.g., wireless power and/or data signals).

When stimulator 112 is coupled to tissue 91, and antenna 154 is in the deployed configuration thereof, antenna 154 typically contacts tissue 91. For some applications, antenna 154 applies a force to tissue 91, thereby stabilizing stimulator 112 at the implantation site. For some applications, the force applied by antenna 154 facilitates coupling of anchor 153 to tissue 91 by increasing friction between the anchor and the tissue. FIG. 9C shows stimulator 112 protruding generally orthogonally from the plane of tissue 91. However, the scope of the invention includes stimulator 112 protruding at other angles from tissue 91, e.g., such that the stimulator protrudes less into the heart chamber.

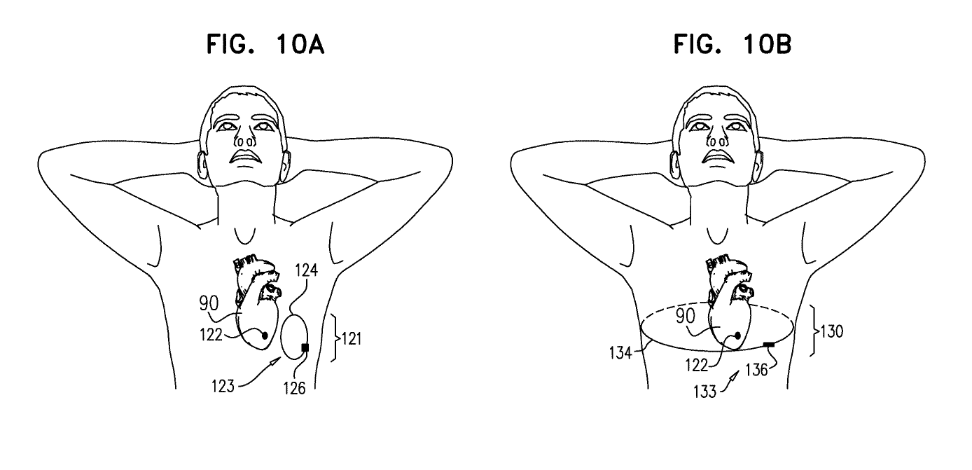

Reference is made to FIGS. 10A-B, which are schematic illustrations of heart-monitoring apparatus, comprising a magnetic element and a detector, in accordance with some applications of the invention. FIG. 10A shows heart-monitoring apparatus 121, comprising a magnetic element, couplable to tissue in a vicinity of heart 90 of the subject, and a detector 123, which comprises a subcutaneously-implantable induction wire 124 and control circuitry 126, connected to the induction wire. A magnetic element 122 is coupled to the tissue, such that beating of the heart moves the magnetic element. Typically, magnetic element 122 is coupled to cardiac tissue, such as by being implanted in a chamber of heart 90. Alternatively, magnetic element 122 is implanted pericardially, or at any other site in which the magnetic element is moved by beating of the heart (including at a site that is not in contact with the heart). Detector 123 is subcutaneously implanted (e.g., in the vicinity of magnetic element 122), such that movement of the magnetic element induces a current in induction wire 124. For example, and as shown in FIG. 10A, detector 123 may be implanted in an anterior subcutaneous region of the thorax. Control circuitry 126 is configured to detect the induced current, and thereby to detect beating of heart 90 and/or a state thereof. For some applications, detector 123 is implanted at a non-subcutaneous site.

Although apparatus 121 is shown in FIG. 10A as comprising one magnetic element 122 and one induction wire 124, it is to be noted that more than one magnetic element and/or induction wire may be used, e.g., to increase sensitivity and/or reliability, and/or to detect more than one movement.

For some applications of the invention, control circuitry 126 comprises control circuitry of a medical device, and is configured to apply a treatment in response to the detected induced current. For example, control circuitry 126 may comprise control circuitry of a pacemaker and/or an ICD, and may be configured to drive a pacing and/or cardioverting-defibrillating current in response to detecting arrhythmia and/or fibrillation of heart 90.

FIG. 10B shows heart-monitoring apparatus 130, comprising magnetic element 122 and a detector 133, which comprises a subcutaneously-implantable induction wire 134 and control circuitry 136, connected to the induction wire. Typically, apparatus 130 (and detector 133 thereof) have functionality similar to that of apparatus 121 (and detector 123 thereof), described with reference to FIG. 10A. Detector 133 is subcutaneously implanted in the vicinity of magnetic element 122, such that movement of the magnetic element induces a current in induction wire 134. For example, and as shown in FIG. 10B, detector 133 may be subcutaneously implanted such that induction wire 134 is subcutaneously disposed around a portion of the subject, such as around the thorax of the subject.

Although apparatus 130 is shown in FIG. 10B as comprising one magnetic element 122 and one induction wire 134, it is to be noted that more than one magnetic element and/or induction wire may be used, e.g., to increase sensitivity and/or reliability.

For some applications of the invention, control circuitry 136 comprises control circuitry of a medical device, and is configured to apply a treatment in response to the detected induced current. For example, control circuitry 136 may comprise control circuitry of a pacemaker and/or an ICD, and may be configured to drive a pacing and/or cardioverting-defibrillating current in response to detecting arrhythmia and/or fibrillation of heart 90.

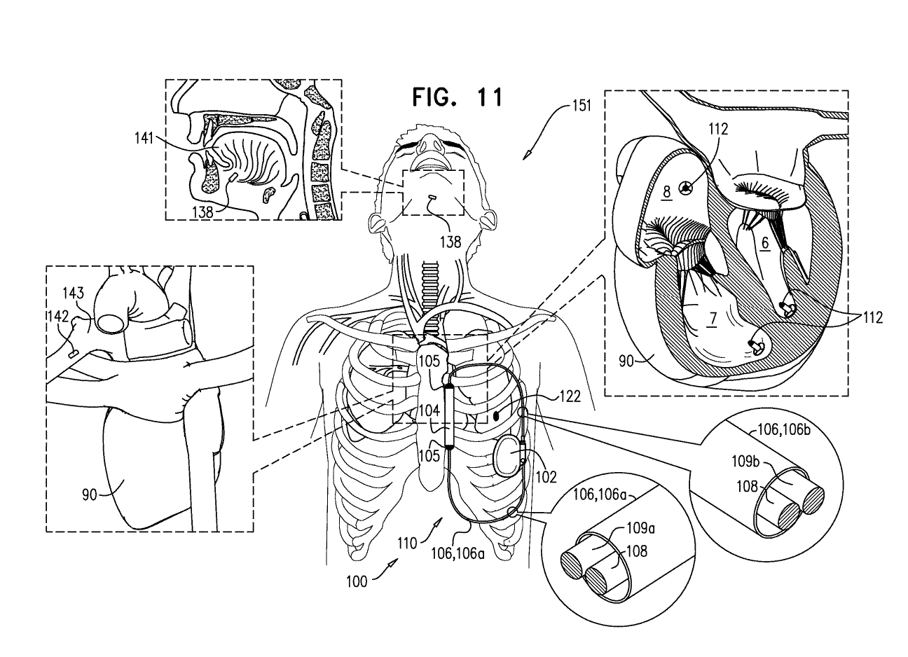

Reference is again made to FIGS. 10A-B. It is noted that, for some applications, magnetic element 122 comprises any magnetic component of a prior art cardiac stimulator, and this cardiac stimulator may be, for example, a leadless pacemaker or a standard pacemaker coupled by a lead to a separate control circuitry. For such applications, detector 123 and/or detector 133 are configured to detect movement of the prior art cardiac stimulator. The scope of the invention thereby includes implanting detector 123 and/or induction wire 124 in a subject in whom a cardiac stimulator is implanted, so as to detect movement of the cardiac stimulator. The scope of the invention further includes implanting one or more magnetic elements 122 at respective sites in the body of the subject that are moved by breathing of the subject, and detecting breathing by detecting movement of the magnetic elements (e.g., as described hereinbelow with reference to FIG. 11).

Reference is made to FIG. 11, which is a schematic illustration of a system 100, comprising a subcutaneous device 110, and at least one cardiac stimulator 112, for treating heart 90 of the subject, in accordance with some applications of the invention.

Device 110 is implantable subcutaneously, and comprises control circuitry 102 and at least one cable 106. Typically, cable 106 is configured to be coupled to control circuitry 102 such that the cable forms a loop outside of the control circuitry. Cable 106 comprises one or more wires disposed therewithin, including at least one power-transmitting wire 108. Typically, power-transmitting wire 108 extends through the full length of cable 106, such that when the cable forms the loop, both ends of the power-transmitting wire connect to control circuitry 102, so as to form a circuit with the control circuitry.

For some applications of the invention, device 110 is provided in an open configuration, in which a proximal cable portion 106a of cable 106 (and, thereby, a proximal end of the power-transmitting wire) is coupled to control circuitry 102, and a distal cable portion 106b of the cable is free. Alternatively, device 110 may be provided with cable 106 completely uncoupled from control circuitry 102. A physician implants cable 106 subcutaneously, such that the cable forms a loop.

The physician subsequently couples, to control circuitry 102, the distal end of the cable (or both ends of the cable, if device 110 is provided with cable 106 completely uncoupled from the control circuitry). That is, subsequently to implanting cable 106, the physician “plugs” the cable into the control circuitry (e.g., into a “header” of the control circuitry), thereby completing the circuit of the power transmitting-wire and control circuitry.

Control circuitry 102 typically comprises a power-storage element (i.e., a power-storage element that is configured to power the control circuitry for a continuous period of greater than 1 hour and/or to store more than 20 mAh), which may be rechargeable (e.g., wirelessly rechargeable) or non-rechargeable. Control circuitry 102 is configured to drive the power-transmitting wire of cable 106 to transmit a wireless power signal, such as a radio-frequency (RF) signal, or a magnetic induction signal. That is, the power-transmitting wire of cable 106 (or the circuit formed thereby) acts as an antenna or induction loop. As described hereinbelow, cardiac stimulator 112 is configured to receive the wireless power signal, and to drive a pacing current into heart 90, at least in part responsively to the power signal.

Typically, device 110 further comprises a cardioversion-defibrillation structure 104, disposed between the proximal and distal portions of cable 106, and comprising one or more electrodes 105. Each electrode 105 is connected to control circuitry 102 by a respective electrode wire 109 (e.g., electrode wire 109a or electrode wire 109b), disposed within cable 106. Typically, cardioversion-defibrillation structure 104 comprises two or more cardioversion-defibrillation electrodes 105, and is configured to drive a cardioverting-defibrillating current therebetween, via tissue of the subject (e.g., as described hereinbelow). For some applications, and as shown in FIG. 11, electrode wire 109a is disposed in cable portion 106a, and electrode wire 109b is disposed in cable portion 106b (i.e., each electrode wire is disposed in a respective cable portion). That is, each electrode wire 109 is disposed in one of the cable portions, and not in the other cable portion. Alternatively, both electrode wires (i.e., electrode wires 109a and 109b) are disposed within one of the cable portions (e.g., within cable portion 106a).

For some applications, cardioversion-defibrillation structure 104 comprises only one electrode 105, and a component (e.g., an outer surface, such as a casing) of control circuitry 102 acts as a second electrode.

Cardiac stimulator 112 is coupled to the heart of the subject, such as by being implanted in a heart chamber of the subject (e.g., as described hereinabove with reference to FIGS. 9A-C). As described hereinabove (e.g., with reference to FIGS. 5A-6C), cardiac stimulator 112 typically comprises components as known for a leadless cardiac stimulator, such as a leadless cardiac pacemaker, e.g., an intraventricular cardiac pacemaker. However, as described hereinabove, stimulator 112 typically comprises no circuitry to directly sense parameters of the heart, and no power-storage element (i.e., no power-storage element that is configured to power the stimulator for a continuous period of greater than 1 hour and/or to store more than 20 mAh). Cardiac stimulator 112 is configured to receive the power signal transmitted by device 110, and, at least in part responsively to the power signal, to drive a current into heart 90. For example, device 110 may transmit the wireless power signal as a series of pulses, which are received by stimulator 112, which drives a pacing current into the heart upon receipt of each pulse. That is, device 110 is configured to drive stimulator 112 to apply the current (e.g., the pacing current) to the heart, by transmitting the wireless power signal.

System 100 is typically configured to detect one or more factors associated with a state of the heart of the subject (e.g., cardiac rhythm or arrhythmia), and to responsively apply an electrical current to the heart of the subject. Non-limiting examples of such functionality, which may be combined with each other and/or with other examples of such functionality, are included here:
For some applications of the invention, (1) control circuitry 102 is configured to detect arrhythmia of the heart of the subject, and to responsively drive the power-transmitting wire of cable 106 to transmit the wireless power signal, and (2) stimulator 112 is configured to responsively apply a pacing current to the heart of the subject. For example, control circuitry 102 may be configured to detect the arrhythmia by detecting electrical signals, such as ECG signals (e.g., a QRS complex thereof). Alternatively or additionally, control circuitry 102 may be configured to detect the arrhythmia by magnetically detecting movement of stimulator 112, implanted in the heart of the subject (e.g., as described with reference to FIGS. 10A-B, mutatis mutandis).
For some applications of the invention, (1) stimulator 112 is configured to detect arrhythmia of the heart of the subject, and (2) stimulator 112 is configured to responsively apply a pacing current to the heart of the subject. For example, stimulator 112 may be configured to detect the arrhythmia by detecting changes in the wireless power signal (e.g., in an amplitude thereof), such as changes caused by movement of the stimulator with respect to device 110 (e.g., with respect to cable 106 thereof). Alternatively or additionally, stimulator 112 may be configured to detect the arrhythmia by detecting electrical signals in the heart of the subject, e.g., by using electrically-conducting anchor 153 and/or second electrode 158. For some applications, stimulator 112 may transmit a wireless feedback signal in response to the detected arrhythmia, e.g., in response to which control circuitry 102 may alter one or more characteristics of the power signal (such as timing of the power signal).
For some applications of the invention, device 110 is configured to detect cardiac fibrillation, and to responsively drive a component of system 100 to apply a cardioverting-defibrillating current to the heart of the subject. For example, device 110 may be configured to detect the fibrillation (e.g., electrically and/or magnetically, as described hereinabove for detecting arrhythmia), and to responsively drive electrodes 105 to apply a cardioverting-defibrillating current to the heart of the subject, so as to cardiovert and/or defibrillate the heart. Alternatively or additionally, device 110 may be configured to transmit a second wireless power signal, in response to which cardiac stimulator 112 is configured to apply the cardioverting-defibrillating current to the heart.

For some applications in which device 110 is configured to (1) transmit the wireless power signal which drives stimulator 112 to drive the pacing current, and to (2) drive the cardioverting-defibrillating current via electrodes 105, device 110 comprises a modified S-ICD in which an additional cable portion couples the control circuitry of the S-ICD to the cardioversion-defibrillation electrode of the S-ICD, thereby forming a closed loop. For such an application, the circuitry and/or sensors of the S-ICD are typically modified so as to facilitate the functionality described hereinabove.

As shown in FIG. 11, system 100 may comprise a plurality of cardiac stimulators 112. For example, stimulators 112 may be implanted in left ventricle 6 of the heart, right ventricle 7 of the heart, and/or an atrium (e.g., right atrium 8) of the heart. For example, implantation of a first stimulator in the right ventricle of the heart, and a second stimulator in the left ventricle of the heart, facilitates cardiac resynchronization therapy (CRT; e.g., biventricular pacing of the heart). Application of pacing current by each stimulator 112 is coordinated with respect to the application of pacing current by the other stimulators. Typically, this coordination is facilitated by control circuitry 102. For example, control circuitry 102 may independently drive each stimulator 112 according to a pre-defined program, and/or in response to one or more factors detected by the control circuitry and/or the stimulators, e.g., as described hereinabove (such as detected cardiac electrical activity and/or detection of movement of the stimulators by magnetic detection and/or by detection of changes in power signal amplitude). Alternatively or additionally, each stimulator 112 may wirelessly communicate directly with one or more other stimulators, so as to facilitate the coordination.

For some applications of the invention, a tongue-muscle stimulator 138 is coupled to a tongue 141 of the subject (e.g., by being implanted sublingually). Stimulator 138 is configured to apply a current to the tongue of the subject in response to receiving a signal from device 110. Stimulator 138 may comprise a power-storage element and/or may wirelessly receive power from device 110, e.g., as described for stimulator 112. Use of tongue-muscle stimulator 138 is described hereinbelow. For some applications, apparatus and methods described in US 2012/0123498 to Gross, for use of a tongue-muscle stimulator, are combined with apparatus and methods described herein.

For some applications of the invention, a pressure sensor 142 is implanted in a pulmonary artery 143 of the subject (or a branch thereof), and is configured to detect wedge pressure, and to responsively transmit a wireless signal in response to the detected wedge pressure. Sensor 142 may comprise a power-storage element and/or may wirelessly receive power from device 110, e.g., as described for stimulator 112.

For some applications of the invention, at least one magnetic element 122 is implanted at a site in the subject that moves when the subject breathes. For example, and as shown in FIG. 11, magnetic element 122 may be placed subcutaneously at a site that is circumscribed by the loop formed by device 110. Alternatively or additionally, magnetic element 122 may be placed subcutaneously elsewhere on the torso of the subject, such as subcutaneously on the back of the subject. For such applications, device 110 is configured to detect breathing of the subject by detecting movement of magnetic element 122. Alternatively, a breathing sensor (not shown), such as an implantable or external breathing sensor, may be placed in communication with device 110 (e.g., with control circuitry 102), such that device 110 is configured to detect breathing of the subject by receiving a signal from the breathing sensor. An example of an external breathing sensor is described in WO 2012-066532 to Gross. For some applications in which the breathing sensor comprises an implantable breathing sensor, the implantable breathing sensor is powered by control circuitry 102.

Congestive heart failure (CHF), cardiac arrhythmia, and/or sleep apnea often present together in an individual subject. For some applications, a system 151, comprising system 100, tongue-muscle stimulator 138, pressure sensor 142, and magnetic element 122, is implanted in such a subject, and control circuitry 102 is configured, in response to detected breathing, wedge pressure, arrhythmia, and/or fibrillation, to drive tongue-muscle stimulator 138, cardiac stimulators 112, and/or electrodes 105 to apply current to the tongue and/or heart of the subject. Thereby, system 151 is configurable to coordinate treatment of congestive heart failure, arrhythmia, fibrillation, and/or sleep apnea.

That is, subcutaneous device 110 (e.g., control circuitry 102 thereof) is configured to be placed in wireless communication with one or more other implanted devices. The other implanted devices typically comprise one or more cardiac stimulators 112, and may also comprise other stimulators (e.g., tongue-muscle stimulator 138), and/or sensors (e.g., breathing sensors and/or pressure sensor 142).

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising a defibrillator, which comprises:
(a) a garment, shaped to be worn around a chest of a patient;
(b) cardiac sensing electrodes, each of which is configured for placement in external or internal contact with the chest of the patient to sense cardiac activity of the patient;
(c) defibrillation electrodes, (i) each of which is coupled to the garment for external placement near the chest of the patient for applying one or more defibrillation shocks, and (ii) which comprise (A) respective reservoirs containing electrically-conductive gel, and (B) respective liners removably covering the gel;
(d) one or more exposure actuators, which are configured to remove the liners from the gel, and which comprise respective motors and rollers; and
(e) control circuitry, which is configured to:
detect cardiac arrhythmia using the cardiac sensing electrodes,
ascertain that the detected cardiac arrhythmia is a type of cardiac arrhythmia that can be treated by applying defibrillation, and
upon ascertaining that the detected cardiac arrhythmia can be treated by applying defibrillation, (i) drive the one or more exposure actuators to remove the liners from the gel, and, thereafter, (ii) drive the defibrillation electrodes to apply at least one defibrillation shock to the chest of the patient.

2. The apparatus according to claim 1, wherein the one or more exposure actuators are configured to remove the liners from the gel by peeling or sliding the liners off of the gel.

3. (canceled)

4. The apparatus according to claim 1, further comprising one or more disposable single-use cartridges, each of which comprises one or more of the defibrillation electrodes, one or more of the liners, one or more of the reservoirs containing the gel, and one or more of the exposure actuators, and the garment is shaped so as to define one or more receptacles for receiving the one or more disposable single-use cartridges.

5. The apparatus according to claim 1, wherein the garment is selected from the group consisting of: a vest and a shirt.

6. Apparatus comprising a defibrillator, which comprises:
(a) a garment, shaped to be worn around a chest of a patient;
(b) cardiac sensing electrodes, each of which is configured for placement in external or internal contact with the chest of the patient to sense cardiac activity of the patient;
(c) defibrillation electrodes, each of which is coupled to the garment for external placement near the chest of the patient for applying one or more defibrillation shocks;
(d) a tightening mechanism, which is configured to modulate a tightness of the defibrillation electrodes against the chest of the patient; and
(e) control circuitry, which is configured to:
detect cardiac arrhythmia using the cardiac sensing electrodes,
ascertain that the detected cardiac arrhythmia is a type of cardiac arrhythmia that can be treated by applying defibrillation, and
upon ascertaining that the detected cardiac arrhythmia can be treated by applying defibrillation, (i) drive the tightening mechanism to increase the tightness of the defibrillation electrodes against the chest of the patient, and (ii) drive the defibrillation electrodes to apply at least one defibrillation shock to the chest of the patient.

7. The apparatus according to claim 6,
wherein the tightening mechanism is configured to modulate the tightness of the defibrillation electrodes against the chest of the patient by modulating a tightness of the garment around the chest of the patient, and
wherein the control circuitry is configured to, upon ascertaining that the detected cardiac arrhythmia can be treated by applying defibrillation, drive the tightening mechanism to increase the tightness of the garment around the chest of the patient.

8. The apparatus according to claim 6, wherein the tightening mechanism comprises one or more balloons, which are configured to push the defibrillation electrodes against the chest of the patient upon inflation of the balloons.

9. The apparatus according to claim 6, wherein the tightening mechanism comprises one or more springs.

10. The apparatus according to claim 6,
wherein the defibrillator further comprises one or more reservoirs containing electrically-conductive gel, and
wherein the tightening mechanism is configured, when driven to increase the tightness, to release the gel to a region between the defibrillation electrodes and the chest to improve electrical conduction during applying of the at least one defibrillation shock.

11. The apparatus according to claim 6,
wherein the defibrillator further comprises one or more reservoirs containing electrically-conductive gel, and
wherein the tightening mechanism is configured, when driven to increase the tightness, to apply pressure to the one or more reservoirs, which releases the gel between the defibrillation electrodes and the chest to improve electrical conduction during applying of the at least one defibrillation shock.

12. The apparatus according to claim 6,
wherein the defibrillation electrodes further comprise (a) respective reservoirs containing electrically-conductive gel, and (b) respective liners removably covering the gel, and
wherein the defibrillator comprises one or more exposure actuators, which are configured to remove the liners from the gel.

13. The apparatus according to claim 12, wherein the one or more exposure actuators are configured to remove the liners from the gel by peeling or sliding the liners off of the gel.

14. The apparatus according to claim 12, wherein the one or more exposure actuators comprise respective motors and rollers.

15. The apparatus according to claim 12, further comprising one or more disposable single-use cartridges, each of which comprises one or more of the defibrillation electrodes, one or more of the liners, one or more of the reservoirs containing the gel, and one or more of the exposure actuators, wherein the garment is shaped so as to define one or more receptacles for receiving the one or more disposable single-use cartridges.

16. The apparatus according to claim 6, wherein the garment is selected from the group consisting of: a vest and a shirt.

17. Apparatus comprising a defibrillator, which comprises:
(a) a garment, shaped to be worn around a chest of a patient;
(b) cardiac sensing electrodes, each of which comprises an electrically exposed surface that is coupled to an inner surface of the garment for external placement on skin of the chest of the patient to sense cardiac activity of the patient;
(c) defibrillation electrodes, each of which is coupled to the garment for external placement near the chest of the patient for applying one or more defibrillation shocks;
(d) a tightening mechanism, which is configured to modulate a tightness of the cardiac sensing electrodes against the chest of the patient; and
(e) control circuitry, which is configured to:
detect the cardiac activity of the patient using the cardiac sensing electrodes, make a preliminary detection of cardiac arrhythmia based on the detected cardiac activity, and ascertain that the preliminarily-detected cardiac arrhythmia is a type of cardiac arrhythmia that can be treated by applying defibrillation, and
upon ascertaining that the preliminarily-detected cardiac arrhythmia can be treated by applying defibrillation:
drive the tightening mechanism to increase the tightness of the cardiac sensing electrodes against the chest of the patient,
thereafter, again detect the cardiac activity of the patient using the tightened cardiac sensing electrodes, and make an improved detection of the cardiac arrhythmia based on the again-detected cardiac activity,
ascertain that the detected cardiac arrhythmia is a type of cardiac arrhythmia that can be treated by applying defibrillation, and
upon ascertaining that the cardiac arrhythmia detected by the improved detection can be treated by applying defibrillation, drive the defibrillation electrodes to apply at least one defibrillation shock to the chest of the patient.

18. The apparatus according to claim 17,
wherein the tightening mechanism is configured to modulate the tightness of the cardiac sensing electrodes against the chest of the patient by modulating a tightness of the garment around the chest of the patient, and
wherein the control circuitry is configured to, upon ascertaining that the preliminarily-detected cardiac arrhythmia can be treated by applying defibrillation, drive the tightening mechanism to increase the tightness of the garment around the chest of the patient.

19. The apparatus according to claim 17, wherein the tightening mechanism comprises one or more balloons, which are configured to push the cardiac sensing electrodes against the chest of the patient upon inflation of the balloons.

20. The apparatus according to claim 17, wherein the tightening mechanism comprises one or more springs.

21-177. (canceled)
